(19) United States
(12) Patent Application Publication
(10) Pub. No.: US 2012/0040905 A1
(43) Pub. Date: Feb. 16, 2012

(54) MEANS FOR PURIFYING A COAGULATION PROTEIN, AND METHODS FOR IMPLEMENTING SAME

(75) Inventors: Gerald Perret, Choisy Le Roi (FR); Michel Nogre, Vanves (FR)

(73) Assignee: LFB BIOTECHNOLOGIES, Les Ulis (FR)

(21) Appl. No.: 13/201,722

(22) PCT Filed: Feb. 19, 2010
(86) PCT No.: PCT/FR10/50293

§ 371 Date: Aug. 16, 2011

(30) Foreign Application Priority Data
Feb. 19, 2009 (FR) 0951081

(51) Int. Cl. A61K 38/36; C07H 21/04; C07K 14/745; C07K 1/14; C08G 81/00; C12N 11/00; C12N 9/64; A61P 7/04; C08G 81/02

(52) U.S. Cl. 514/13.7; 536/23.1; 530/381; 525/54.2; 435/174; 435/226

(57) ABSTRACT

An affinity substrate for selectively binding a coagulation protein, includes a substrate solid on which nucleic aptamers binding specifically to the coagulation protein are immobilized.

### FIELD OF THE INVENTION

The present invention relates to the field of the purification of coagulation proteins that can be used as active ingredients of medicaments.

### PRIOR ART

Coagulation proteins have for a long time constituted active ingredients for medicaments. Among the coagulation proteins used as active ingredients of medicaments, mention may in particular be made of factor VII, factor VIII, thrombin or else von Willebrand factor.

Until recently, coagulation proteins were exclusively obtained by purification from human plasma. Over the past few years, some coagulation proteins have been obtained in the form of recombinant proteins produced in body fluids of transgenic mammals, for example in the milk of transgenic mammals.

In all cases, the starting material containing the coagulation protein to be purified consists of a material of complex constitution, in which the protein of interest is present in combination with a very large number of substances, including proteins, lipids, carbohydrates, amino acids, mineral salts, cell debris and metabolic waste such as urea, uric acid or bilirubin.

The development of a method for purifying a coagulation protein is therefore a complex task, given the health and regulatory characteristics required for the marketing of a medicament for human use.

It is understood that, in a medicament intended for the prevention or treatment of coagulation disorders, the coagulation protein used as active ingredient should be present in a highly purified form and not be associated with undesirable substances capable of being detrimental to the organism, including other coagulation proteins, albumin, immunoglobulins, or else degradation products of the above-mentioned proteins.

Methods for purifying various coagulation proteins are known, including methods for purifying factor VIII, anti-thrombin-III, plasminogen, factor VII or else von Willebrand factor.

All the known methods comprise a succession of selective-separation steps based on steps of protein precipitation, of passage over chromatography substrates followed by sequential-elution steps, deep-filtration steps, ultrafiltration steps or else concentration steps.

It is specified that the development of methods for purifying coagulation proteins, whether these methods are entirely novel, or whether these methods are adaptations, even minimal adaptations, of known methods, requires long research and numerous controls of conformity of the successive intermediate products, in order to be sure that the final product will be obtained with a great purity, in a nonmodified form, and substantially free of undesirable substances. In particular, for coagulation proteins having an enzymatic activity, for instance factors VII, VIII and XI, it is essential that the purified final protein not be activated. However, the activation of a coagulation protein is liable to be induced during any one of the purification steps, including during the chromatography steps, if predetermined set conditions, for example quality of the filters or of the chromatographic substrate used, salt concentration or else temperature, are not adhered to.

The aforementioned at least partially explains why the known methods do not comprise an affinity chromatography step, based on the principle of specific immobilization of the coagulation protein of interest on a ligand grafted to the chromatographic substrate, and then recovery of the purified protein in the chromatography eluate.

The absence of an affinity chromatography purification step in methods for purifying proteins of therapeutic interest is also explained by the drawbacks of this technique in which detachment of a part of the ligand molecules grafted to the affinity substrate is observed, said ligand molecules being found associated with the purified therapeutic protein in the volume of the eluate. It is understood that the presence, in a medicament, for treating coagulation disorders, of constituent substances of a chromatography substrate, which are possibly detrimental to the organism, is prohibited by the medical regulations.

Although the known methods for purifying coagulation proteins are satisfactory, there is a need in the prior art for alternative methods or methods that are improved compared with the existing methods.

### SUMMARY OF THE INVENTION

The invention relates to an affinity substrate for selectively binding a coagulation protein, comprising a solid substrate material on which nucleic aptamers which bind specifically to said coagulation protein are immobilized.

In some embodiments, said nucleic aptamers consist of deoxyribonucleic aptamers.

A subject of the invention is also a method for immobilizing a coagulation protein on a substrate, comprising a step during which a sample containing said coagulation protein is brought into contact with an affinity substrate.

The invention also relates to a method for purifying a coagulation protein, comprising the following steps:
a) bringing a sample containing a coagulation protein into contact with an affinity substrate as defined above, in order to form complexes between (i) the nucleic aptamers immobilized on said affinity substrate and (ii) said coagulation protein, and
b) releasing the protein from the complexes formed in step a), and
c) recovering said coagulation protein in a purified form.

It also relates to a purified composition of a recombinant human coagulation protein comprising at least 99.9% by weight of said recombinant human protein and which is substantially free of nonhuman proteins.

### DESCRIPTION OF THE FIGURES

FIG. 1 illustrates a chromatography profile obtained during the implementation of the method for purifying a recombinant human factor VII produced in rabbit milk, with the affinity substrate in which anti-human FVII nucleic aptamers are immobilized. Along the x-axis: the time; along the y-axis: the absorbance value (OD) at 254 nanometers.

FIG. 2 represents the curve of the values of the measurement of absorbance at 280 nm as a function of time.

FIG. 3 is an image of an SDS PAGE electrophoresis gel with coomassie blue staining enabling relative quantification of the bands. From the left to the right of the gel in FIG. 3, the lanes represent the results of migration of the following starting products, from left to right of the figure:
lane “2” or “MD”: the starting composition Betafact®,
lane “3” or “NR”: the nonretained fraction corresponding to peak No. 1 of the chromatographic profile of FIG. 2,
lane “4” or “E1”: the elution fraction corresponding to peak No. 2 of the chromatographic profile of FIG. 2.

FIG. 4 represents the curve of the values of the measurement of absorbance at 280 nm as a function of time.

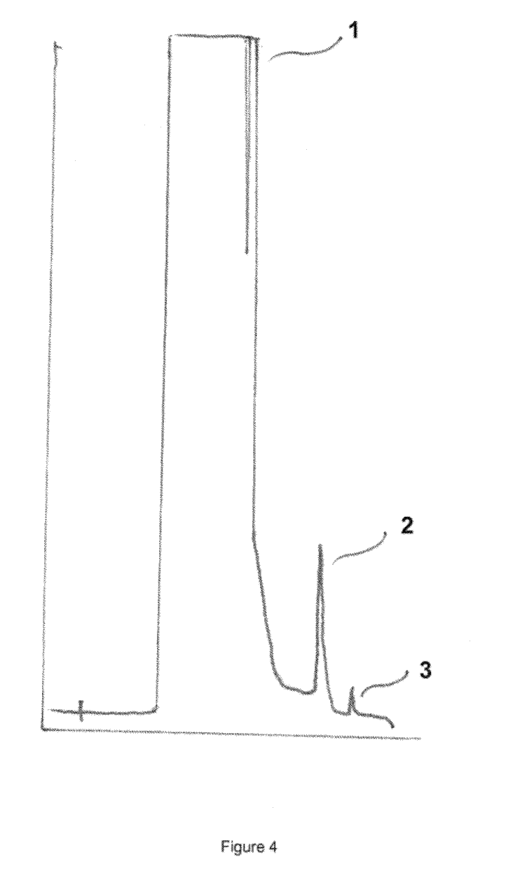

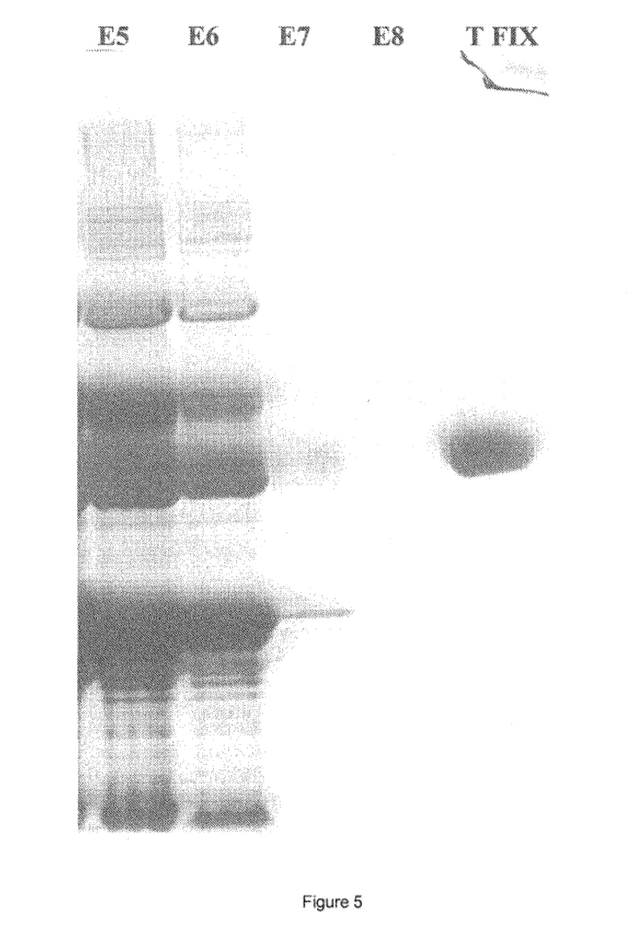

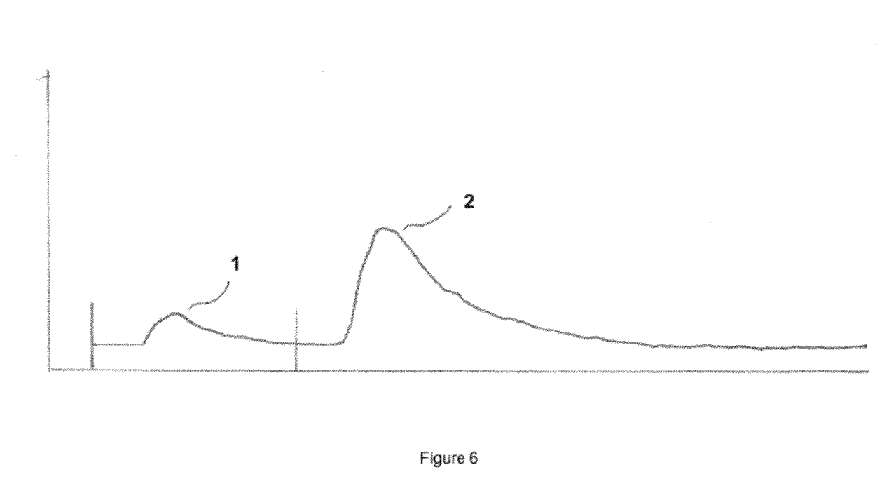

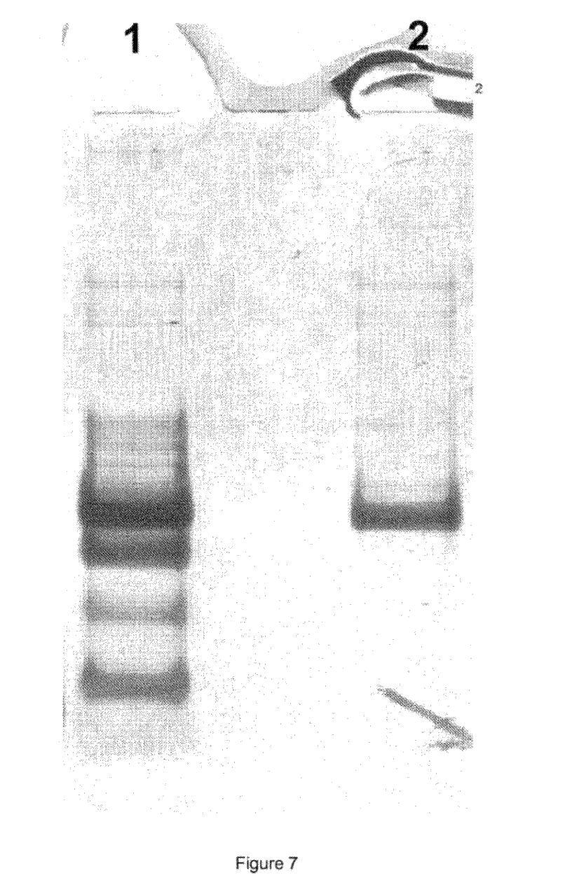

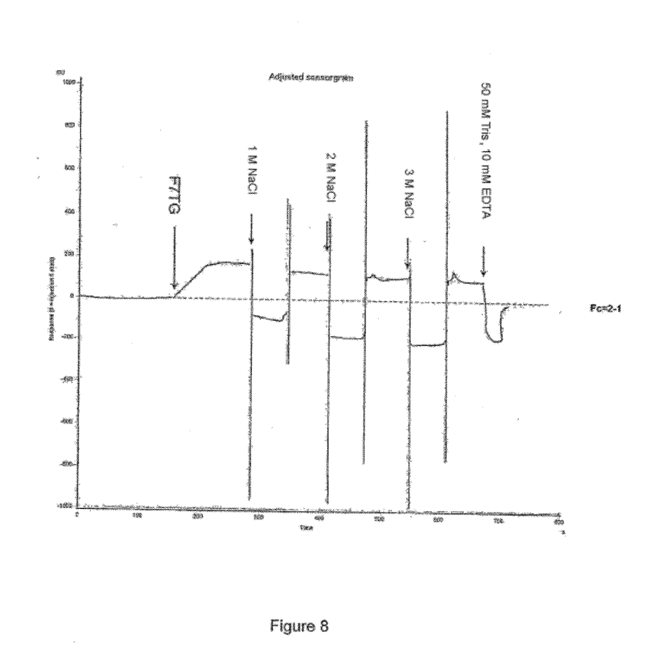

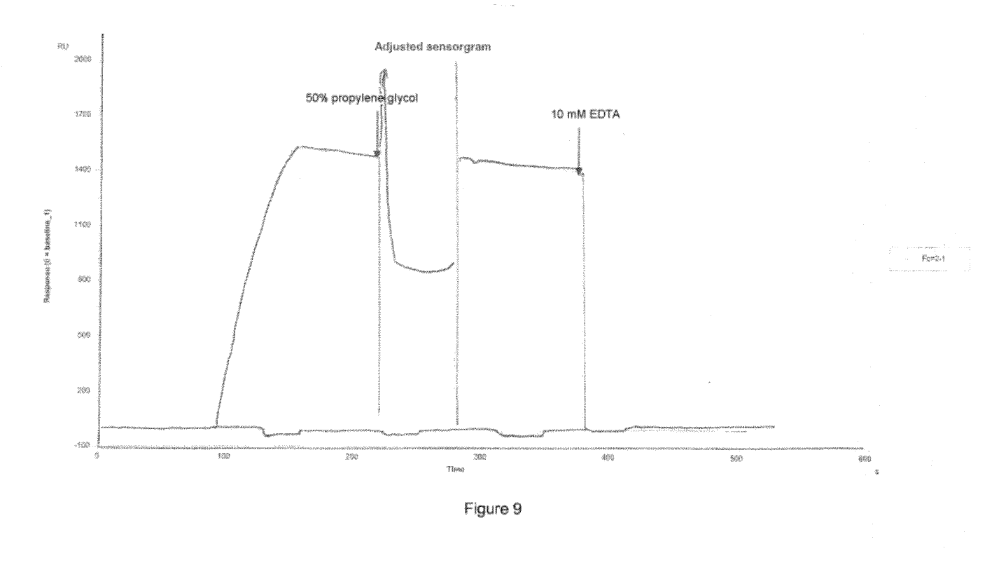

FIG. 5 is an image of an SDS PAGE electrophoresis gel. From the left to the right of the gel in FIG. 5, the lanes represent the results of migration of the following starting products:
lane “E5”: the starting composition of transgenic sow's milk containing transgenic human factor IX prepurified by means of an MEP HyperCel chromatographic step,
lane “E6”: the nonretained fraction corresponding to peak No. 1 of the chromatographic profile of FIG. 4,
lane “E7”: the elution fraction corresponding to peak No. 2 of the chromatographic profile of FIG. 4,
lane “E8”: the regeneration fraction corresponds to peak No. 3 of the chromatographic profile of FIG. 4,
lane “T FIX”: purified human plasma factor IX control.

FIG. 6 represents the curve of the values of the measurement of absorbance at 280 nm as a function of time. In FIG. 6, peak No. 1 corresponds to the fraction of the starting product which was not retained on the column. Peak No. 2 corresponds to the elution fraction.

FIG. 7 represents an image of a gel of the starting product and also the eluted product, analyzed by SDS PAGE with silver nitrate staining in order to visualize the elimination of the impurities: lane No. 1 corresponds to the starting-product fraction and lane No. 2 to the elution fraction. Despite the considerable purity of the starting product, it is noted that the eluted fraction no longer contains any contaminants or degraded forms.

FIG. 8 represents the curves of binding of the Mapt2 immobilized aptamer to the recombinant human factor VII produced in the milk of a transgenic rabbit. The arrows correspond to the time of the various injections, respectively from left to right on FIG. 8: 1: injection of recombinant factor VII; 2: injection of a buffer containing 1M NaCl; 3: injection of a buffer containing 2M NaCl; 4: injection of a buffer containing 3M NaCl; 5: injection of a 50 mM Tris buffer containing 10 mM EDTA. Along the x-axis: the time expressed in seconds; along the y-axis: the value of the response signal, expressed in arbitrary units (RU).

FIG. 9 represents the curves of binding of the Mapt2 immobilized aptamer to the recombinant human factor VII produced in the milk of a transgenic rabbit. The arrows correspond to the time of the various injections, respectively from left to right on FIG. 9: 1: 50% propylene glycol; 2: EDTA at 10 mM.

### DETAILED DESCRIPTION OF THE INVENTION

After long research, the applicant has developed a method for purifying coagulation proteins, comprising a step of chromatography during which there is specific binding of the protein of interest to a ligand preimmobilized on the chromatography substrate, and then of release of the protein of interest retained on said substrate and recovery of the purified protein in the eluate volume.

More specifically, according to the invention, a method is provided for purifying coagulation proteins, comprising a step of specific binding of the coagulation protein of interest to a substrate on which nucleic aptamers which bind specifically to said protein of interest are immobilized, followed by a step of recovery of said purified protein of interest.

Surprisingly, it has been shown, according to the invention, that affinity chromatography substrates on which are immobilized nucleic aptamers specific for the coagulation protein of interest, i.e. including in the form of immobilized compounds comprising such nucleic aptamers, can be used successfully in methods for obtaining coagulation proteins that can be used as active ingredients of a medicament.

The present invention relates to an affinity substrate for selectively binding a coagulation protein, comprising a solid substrate material on which nucleic aptamers which bind specifically to said coagulation protein, including in the form of compounds comprising such nucleic aptamers, are immobilized.

The molecules of nucleic aptamer which bind specifically to the coagulation protein of interest, and also the compounds comprising such nucleic aptamers, constitute sites for specific binding of said target protein which are carried by said solid substrate material.

The term “nucleic aptamers” encompasses single-stranded nucleic acids having a length of 5 to 120 nucleotides and which bind specifically to a coagulation protein. The expression “compounds comprising a nucleic aptamer” also encompasses compounds which comprise, in their structure, said nucleic acids defined above. Thus, the compounds comprising a deoxyribonucleic acid which binds specifically to a human or mammalian coagulation protein encompass compounds in which said nucleic acid is included in a structure comprising a biotin molecule.

It has been shown, according to the invention, that an affinity substrate as defined above allows efficient immobilization of the coagulation protein of interest, which may also be referred to as “target protein” in the present description.

An affinity substrate as defined above has a high capacity for adsorption of the target protein, since bringing the solid substrate into contact with a liquid solution containing the coagulation protein to be purified makes it possible to saturate at least 50 percent of the target sites carried by the solid substrate.

It has also been shown, according to the invention, that the coagulation protein molecules which are specifically bound to the nucleic aptamer molecules can subsequently be eluted from the affinity substrate with a good yield, of at least 75 percent.

Furthermore, it has been shown that the affinity substrate of the invention has a high specificity for the coagulation protein of interest, since said coagulation protein can be found with a degree of purity ranging up to 99.95 percent by weight, relative to the total weight of the proteins contained in the eluate.

As is illustrated in the examples, an increase of two orders of magnitude in the purity of the coagulation protein of interest can further be obtained with an affinity substrate according to the invention, using, as starting product, a composition comprising the target coagulation protein at a high degree of purity, for example at more than 98% by weight, relative to the total weight of the proteins contained in said starting product. It is specified that such an increase in purity is obtained including when the impurities present in the starting product have structural or physicochemical characteristics very close to those of the target coagulation protein that it is desired to purify.

It has also been shown that the affinity substrate of the invention makes it possible to purify coagulation proteins of primary sequence which are very distinct, such as human factor VII and human factor IX.

It has also been shown that the affinity substrate of the invention makes it possible to purify coagulation proteins from starting media of complex composition, such as a plasma composition enriched in human factor IX and a milk from an animal that is transgenic for human factor IX, with great specificity of binding to said coagulation protein of interest.

It has also been shown that the affinity substrate of the invention makes it possible to selectively purify a human coagulation protein from a starting medium also comprising a nonhuman orthologous protein, for example from a milk of an animal that is transgenic for human factor IX, said milk also comprising factor IX produced naturally by said transgenic animal, it being possible for said factor IX produced naturally by said transgenic animal (or endogenous FIX) to be, for example, a porcine FIX.

It has also been shown that the affinity substrate of the invention makes it possible to selectively purify a human coagulation protein from a starting medium also comprising a nonhuman orthologous protein, for example from a milk of an animal that is transgenic for human factor VII, said milk also comprising factor VII produced naturally by said transgenic animal, it being possible for said factor VII produced naturally by said transgenic animal (or endogenous FVII) to be, for example, a rabbit FVII.

Importantly, it has been shown that the above characteristics of high absorption capacity, of good yield and of high specificity of the affinity substrate of the invention are obtained in particular for the purification of the coagulant protein of interest from a starting medium of complex composition, such as blood plasma or else a biological fluid of a mammal that is transgenic for said coagulant protein of interest.

It has also been shown that the immobilization of the nucleic aptamers on the solid substrate material is irreversible and long-lasting, since the presence of nucleic aptamers detached from the substrate is not detectable in the eluate solution.

In particular, it has been shown that the affinity substrate of the invention can be “regenerated” by elimination of the proteins that have remained bound to the substrate after elution, very many times without significant impairment of (i) its capacity for absorption of the target coagulation protein, (ii) its specificity with respect to said target protein, or else (iii) its absence of release of the nucleic aptamers immobilized on the solid substrate material. Furthermore, the regeneration of the affinity substrate of the invention can be carried out according to known techniques and with known regenerating agents, such as urea.

The nucleic acids used as ligands of the coagulation protein of interest have many advantages. By virtue of their oligonucleotide nature, the aptamers have a weak immunogenicity and a high resistance to stringent physicochemical conditions (presence of urea, of DMSO, of a very acidic or very basic pH, use of organic solvents or of a high temperature) which allow varied strategies for controlling health safety, in particular safety with respect to viruses or nonconventional pathogenic agents, in the context of use as an affinity ligand. In addition, they are highly selective. Finally, as already mentioned, the production of aptamers involves relatively limited costs. Thus, the combination of above characteristics of the affinity substrate of the invention bring about the ability of said affinity substrate to be used as a means for purifying a coagulation protein, since
said affinity substrate makes it possible to carry out methods for purifying a coagulation protein having a very high degree of purity,
said affinity substrate does not detectably release undesirable substances, in particular nucleic aptamer molecules, into the solution of eluate containing the purified coagulation protein,
the possible detachment of nucleic aptamer molecules does not lead to drawbacks for human health,
the binding and then the elution of the coagulation protein of interest on the affinity substrate, when it is a coagulation protein with enzymatic activity, does not lead to the formation of detectable amounts of the activated form of said coagulation protein,
said affinity substrate is relatively inexpensive to produce, in particular owing to the low costs of nucleic aptamer production, and
the use of said affinity substrate for purifying a coagulation protein is itself relatively inexpensive owing to the longevity of said substrate, due in particular to the possibility of regenerating it many times, and over a long period of time.

In addition, as has already been mentioned, the affinity substrate of the invention is capable of selectively binding, in a reversible manner, the target coagulation protein with a good yield and good specificity, in methods of purification from media of complex constitution, in particular from media conventionally used on the industrial scale, such as human plasma or culture media or biological fluids containing said protein of interest.

Furthermore, as will be detailed later in the present description, the affinity substrate of the invention is suitable for the treatment of large volumes of solution containing the coagulation protein of interest. The affinity substrate of the invention thus constitutes a tool for purifying a coagulation protein which is perfectly suitable for use on the industrial scale.

Other advantages of the affinity substrate according to the invention will subsequently be specified in the present description, either in relation to the description of the affinity substrate itself, or in relation to the methods for purifying a coagulation protein in which said affinity substrate is used.

To the applicant's knowledge, the present invention describes for the first time an affinity substrate comprising immobilized nucleic aptamers, for purifying a coagulation protein under conditions for use on the industrial scale. A variety of nucleic aptamers specific for thrombin, for factor VII and for factor IX is, moreover, known in the prior art (see PCT Application No. WO 02/26932).

The term “selective binding” is intended to mean, for the purposes of the present description, the specific noncovalent binding of the coagulation protein of interest to the constituent immobilized nucleic acids of the affinity substrate, which is reversible by bringing the affinity substrate, on which noncovalent complexes between said nucleic acids and said protein of interest are formed, into contact with a solution of suitable composition, which can also be referred to as elution solution.

The term “coagulation protein” is intended to mean, according to the invention, any protein involved in the chain of cascade reactions resulting in the formation of a blood clot. The coagulation proteins encompass factor I (fibrinogen), factor II (prothrombin), factor V (proaccelerin), factor VII (proconvertin), factor VIII (anti-hemophilic factor A), factor IX (anti-hemophilic factor B), factor X (Stuart factor), factor XI (Rosenthal factor or PTA), factor XII (Hageman factor), factor XIII (fibrin-stabilizing factor or FSF), PK (Prekallikrein), HMWK (high-molecular-weight kininogen), tissue thromboplastin, heparin cofactor II (NCII), protein C (PC), thrombomodulin (TM), protein S (PS), von Willebrand factor (vWF) and tissue factor pathway inhibitor (TFPI), or else tissue factors.

In some embodiments, the coagulation protein consists of a coagulation protein with enzymatic activity.

The coagulation proteins with enzymatic activity encompass factor II (prothrombin), factor VII (proconvertin), factor IX (anti-hemophilic factor B), factor X (Stuart factor), factor XI (Rosenthal factor or PTA), factor XII (Hageman factor), factor XIII (fibrin-stabilizing factor or FSF) and PK (Prekallikrein).

In some preferred embodiments, the coagulation protein consists of a natural or recombinant human coagulation protein.

In preferred embodiments, the coagulation protein is natural or recombinant human factor VII.

Generally, the solid substrates on which the aptamers of the invention can be immobilized encompass any type of substrate having the structure and the composition commonly found for filter substrates, membranes, etc. The solid substrates encompass in particular resins, affinity chromatography column resins, polymer beads, magnetic beads, paramagnetic beads, substrate materials of filter membranes, etc. The solid substrates also in particular encompass materials based on glass or on metal, such as steel, gold, silver, aluminum, copper, silicon, glass or ceramic. The solid substrates also in particular encompass polymer materials, such as a polyethylene, a polypropylene, a polyamide, a polyvinylidene fluoride, and combinations thereof.

In some embodiments, the solid substrate may be coated with a material which facilitates attachment, binding, complex formation, immobilization or interaction with the aptamers, or with the compounds comprising said aptamers.

In some embodiments, the solid substrate is a glass slide of which the surface is coated with a layer of gold, with a layer having undergone a treatment by carboxymethylation, with a layer of dextran, of collagen, of avidin, of streptavidin, etc.

In this way, the aptamers according to the invention, or the compounds comprising the aptamers according to the invention, can be immobilized on the solid substrate by means of an attachment coating, as, for example, described above, either by chemical reaction with the creation of covalent bonds, or by association via noncovalent bonds, such as hydrogen bonds, electrostatic forces, Van der Waals forces, etc.

The examples describe embodiments of an affinity substrate according to the invention in which the nucleic aptamers are immobilized, by means of the compounds in the structure of which they are included, via noncovalent bonds to the solid substrate material.

In the examples, affinity substrates comprising a solid substrate material at the surface of which streptavidin molecules are grafted are in particular described, the nucleic aptamers being included in the structure of compounds comprising aptamers coupled, at one of their ends, to a biotin molecule, and said aptamers being immobilized on said solid substrate material by noncovalent immobilization between the streptavidin molecules of the substrate material and the biotin molecules of the compounds comprising said nucleic aptamers.

In the examples, an embodiment of an affinity substrate comprising a substrate material on which streptavidin molecules are grafted is described. Such solid substrate materials are readily commercially available.

The expression “nucleic acids which bind specifically to a coagulation protein”, or “aptamers” or “nucleic aptamers” is intended to mean DNA (deoxyribonucleic acid) or RNA (ribonucleic acid) molecules having the capacity to bind to a given coagulation protein of interest which is greater than the capacity to bind to any other protein.

In some embodiments, the constituent nucleic aptamers of the affinity substrate according to the invention have the capacity to bind to a given human coagulation protein which is greater than the capacity to bind to any other human protein.

In some embodiments, the nucleic aptamers according to the invention have in particular the capacity to bind to a given human coagulation protein, which is greater than the capacity to bind to any other homologous coagulation protein encoded by the genome of a nonhuman mammal. By way of illustration, a nucleic aptamer specific for human factor VII has a capacity to bind to human factor VII which is greater than the capacity to bind to factor VII originating from a nonhuman mammal, including a rabbit factor VII.

For the purpose of the present description, a first nucleic acid has a capacity to bind to human factor VII/VIIa which is greater than that of a second nucleic acid of equivalent mass, when, by using any one of the above binding detection techniques and under the same test conditions, a statistically significant higher binding signal value is obtained with the first nucleic acid, compared with that obtained with the second nucleic acid. By way of illustration, when the binding detection technique used is the Biacore® technique, a first nucleic acid has a capacity to bind to human factor VII/VIIa which is greater than that of a second nucleic acid of equivalent mass, when the resonance signal value for the first nucleic acid, irrespective of the resonance measurement unit expressed, is statistically higher than the resonance signal value measured for the second nucleic acid. Two “statistically” distinct measurement values encompass two values which have, between them, a difference greater than the measurement error of the binding detection technique used.

Preferentially, a nucleic aptamer of an affinity substrate of the invention has a strong affinity for the target coagulation protein. Preferentially, said nucleic aptamer has a Kd value, with respect to said target coagulation protein, of less than 500 nM. The Kd value can be measured according to the Biacore® technique.

By way of illustration, it has been shown that the nucleic aptamer comprising the sequence SEQ ID No 86 has a capacity to bind to human factor VII/VIIa which is significantly greater than its capacity for binding to any factor VII/VIIa originating from a nonhuman mammal. In particular, although a nucleic acid of sequence SEQ ID No 86 has a strong capacity to bind to any type of human factor VII/VIIa, including a natural factor VII/VIIa or a recombinant factor VII/VIIa, it has a weak or zero capacity to bind to a factor VII/VIIa encoded by the genome of a nonhuman mammal, including a rabbit factor VII/VIIa.

More specifically, for the aptamer comprising the nucleic acid of sequence SEQ ID No 86, it has been determined, according to the Biacore® technique, that the value for the capacity for binding to human factor VII/VIIa, expressed as the dissociation constant Kd, is approximately 100 nM. Furthermore, said nucleic aptamer has a binding capacity which is identical both with respect to human plasma factor VII/VIIa and to recombinant human factor VII/VIIa, for example produced in a transgenic rabbit.

It has also been shown that the complexes between a nucleic acid of sequence SEQ ID No 86 and a human factor VII/VIIa are stoichiometric, i.e. the ratio of the number of molecules of nucleic acid of sequence SEQ ID No 86 to the number of molecules of human factor VII/VIIa that are complexed is approximately 1:1, and can be in particular 1:1.

As has already been mentioned above, the affinity substrate of the invention can be used in a step of a method for purifying a recombinant human coagulation protein produced by a nonhuman transgenic mammal. In such embodiments of a method for purifying a coagulation protein, the complex starting medium comprises the recombinant human protein as a mixture with numerous proteins produced naturally by said transgenic mammal, including, as appropriate, the coagulation protein homologous to the recombinant human protein. It is understood that, in such embodiments, it is advantageous for the constituent nucleic aptamers of the affinity substrate of the invention to bind selectively to the human protein of interest, and not to bind, under the same operating conditions, to the homologous protein naturally produced by the nonhuman mammal.

In these particular embodiments of the constituent nucleic aptamers of the affinity substrate of the invention, the capacity of said aptamers to bind “specifically” to the human coagulation protein of interest can also be expressed as the ratio of the dissociation constants Kd, respectively for the human protein and for the nonhuman mammalian homologous protein.

According to yet another characteristic of a constituent nucleic aptamer of the affinity substrate according to the invention, the capacity of said nucleic aptamer to bind specifically to the human coagulation protein can also be expressed by the following condition (A):

human Kd/nonhuman Kd<0.01 (A),

in which:
“human Kd” is the dissociation constant of a nucleic aptamer for said human protein, expressed in molar units, and
“nonhuman Kd” is the dissociation constant of said nucleic aptamer for the nonhuman homologous protein, expressed in the same molar units.

Preferentially, for optimum use of the affinity substrate of the invention in methods for purifying a recombinant human coagulation protein, a nucleic aptamer which binds specifically to said recombinant human protein can also be defined by a human Kd/nonhuman Kd ratio of less than 0.005.

The constituent nucleic aptamers of the affinity substrate of the invention can be prepared according to the technique called SELEX. The term “aptamer” as used encompasses a molecule of single-stranded nucleic acid, DNA or RNA, capable of binding specifically to a protein. Aptamers generally comprise between 5 and 120 nucleotides and can be selected in vitro according to a method known as SELEX (Systematic Evolution of Ligands by Exponential Enrichment), which was initially described in particular in PCT application No. WO 1991/019813. The SELEX method for selecting aptamers consists in bringing a protein into contact with a combinatorial library of nucleic acids, DNA or RNA (in general 1015 molecules); the nucleic acids which do not bind to the target are eliminated, the nucleic acids which bind to the target are isolated and amplified. The method is repeated until the solution is sufficiently enriched with the nucleic acids having a good affinity for the protein of interest (Tuerk and Gold, “Systematic evolution of ligands by exponential enrichment: RNA ligands to bacteriophage T4 DNA polymerase” (1990) Science, 249(4968): 505-10 and Ellington and Szostak, “In vitro selection of RNA molecules that bind specific ligands”, (1990) Nature Aug 30; 346(6287):818-22). Other examples of the SELEX method are given in documents EP 0 786 469, EP 668 931, EP 1 695 978 and EP 1 493 825, the teachings of which can be reproduced in the implementation of the method for selecting a nucleic aptamer used according to the invention.

For its use in a means for purifying human factor VII/VIIa, a nucleic acid which binds specifically to a coagulation protein of interest is preferentially included in a chemical structure, also called “compound” in the present description, which also comprises a spacer means and, where appropriate, a means of immobilization on a solid substrate.

In some embodiments, the nucleic aptamer is included in the structure of a compound of formula (I) below:

[FIX]ₓ-[SPAC]_{y}-[APT] (I),

in which:
[FIX] signifies a compound for immobilization on a substrate,
[SPAC] signifies a spacer chain,
[APT] signifies a nucleic acid which binds specifically to a coagulation protein, also denoted nucleic aptamer,
x is an integer equal to 0 or 1, and
y is an integer equal to 0 or 1.

In some embodiments of the compound of formula (I), [APT] consists of a deoxyribonucleic acid of sequence SEQ ID No 86.

The “spacer chain” denoted [SPAC] in the compound of formula (I) may be of any known type. Said spacer chain has the function of physically distancing the nucleic acid from the surface of the solid substrate on which said compound can be immobilized and of allowing a relative mobility of the nucleic acid relative to the surface of the solid substrate on which it can be immobilized. The spacer chain limits or prevents steric hindrance due to the solid substrate and the nucleic acid being too close to one another, thereby impeding binding events between said nucleic acid and molecules of coagulation protein that may be brought into contact with said nucleic acid.

In the compound of formula (I) the spacer chain is preferentially bonded to the 5′ end or to the 3′ end of the aptamer nucleic acid.

Advantageously, the spacer chain is bonded both to one end of the aptamer and to the solid substrate. This construction with a spacer has the advantage of not directly immobilizing the aptamer on the solid substrate. Preferably, the spacer chain is a nonspecific oligonucleotide or polyethylene glycol (PEG). When the spacer chain consists of a nonspecific oligonucleotide, said oligonucleotide advantageously comprises at least 5 nucleotides in length, preferably between 5 and 15 nucleotides in length.

In the embodiments of a compound of formula (I) in which the spacer chain consists of a polyethylene glycol, said spacer chain encompasses a polyethylene glycol of PEG(C18) type, sold, for example, by the company Sigma Aldrich.

In order to immobilize the aptamer on the spacer chain, the nucleic acid may be chemically modified with various chemical groups, such as groups which make it possible to immobilize said nucleic acid covalently, for instance thiols, amines or any other group capable of reacting with chemical groups present on the solid substrate.

In the compound of formula (I), the compound [FIX] consists of a compound chosen from (i) a compound capable of forming one or more covalent bond(s) with the solid substrate material and (ii) a compound capable of bonding specifically to the solid substrate by means of weak noncovalent bonds, including hydrogen bonds, electrostatic forces or Van der Waals forces.

The first type of compound [FIX] encompasses bifunctional coupling agents, such as glutaraldehyde, SIAB or else SMCC.

The compound SIAB, described by Hermanson G. T. (1996, Bioconjugate techniques, San Diego: Academic Press, pp 239-242), is the compound of formula (I) below:

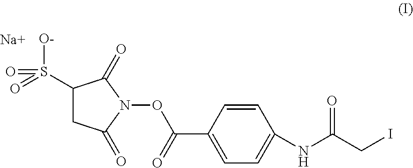

The compound SIAB comprises two reactive groups, respectively an iodoacetate group and a sulfo-NHS ester group, these groups reacting respectively with amino and sulfhydryl groups.

The compound SMCC, which is described by Samoszuk M. K. et al. (1989, Antibody, Immunoconjugates Radiopharm., 2(1): 37-46), is the compound of formula (II) below:

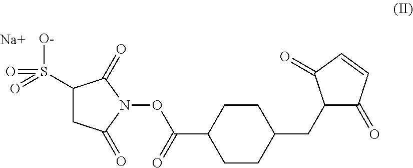

The compound SMCC comprises two reactive groups, respectively a sulfo-NHS ester group and a maleimide group, which react respectively with an amino group and a sulfhydryl group.

The second type of compound [FIX] encompasses biotin, which is capable of binding specifically in a noncovalent manner to avidin or streptavidin molecules present on the mobile substrate.

Once immobilized on the solid substrate via the spacer, the aptamer is advantageously modified at its free end (end not bonded to the spacer) by virtue of, and without being limited thereto, a chemically modified nucleotide (such as 2′-O-methyl- or 2′-fluoropyrimidine, 2′-ribopurine, phosphoramidite), an inverted nucleotide or a chemical group (PEG, polycations, cholesterol). These modifications make it possible to protect the aptamer against enzymatic degradations.

A nucleic acid which binds specifically to a coagulation protein, also denoted nucleic aptamer for the purposes of the present description, is chosen from a DNA and an RNA.

Nucleic aptamers capable of binding to various proteins involved in the blood coagulation pathway are already known in the prior art, including aptamers which bind von Willebrand factor (PCT application No. WO 2008/150495), aptamers which bind alpha-thrombin (European patent application No. EP 1 972 693) or thrombin (Zhao et al., 2008, Anal Chem, Vol. 80(19): 7586-7593), aptamers which bind factor IX/IXa (Subash et al., 2006, Thromb Haemost, vol. 95: 767-771; Howard et al., 2007, Atherioscl Thromb Vasc Biol, vol. 27: 722-727; PCT application No. WO 2002/096926; U.S. Pat. No. 7,312,325), and aptamers which bind factor X/Xa (PCT application No. WO 2002/096926; U.S. Pat. No. 7,312,325).

Nucleic aptamers which bind to human factor VII/VIIa have also been described in the prior art (Rusconi et al., 2000, Thromb Haemost, vol. 84(5): 841-848; Layzer et al., 2007, Spring, vol. 17: 1-11).

It is specified that none of the above aptamers is described for its use for purifying the target protein to which it binds.

Surprisingly, it is shown according to the invention that it is possible to fabricate an affinity substrate as defined in the present description by using, as nucleic aptamers, DNA aptamers, which are nevertheless considered in the prior art to be ligand nucleic acids that are not easy to use, and the specificity of which for the target protein is less than the specificity of the RNA molecule of corresponding sequence. In particular, it is accepted in the prior art that ligand DNAs have less flexibility than the corresponding RNA and that, consequently, they are less capable than the ligand RNAs of undergoing conformational changes. It is recalled that, when a nucleic aptamer binds to the target protein, a conformational change occurs. It has also been described that, the faster the conformational change of the nucleic aptamer, the higher the affinity of said nucleic aptamer for the target protein (Michaud et al., 2003, Anal Chem, vol. 76: 1015-1020; Brumbt et al., 2005, Anal Chem, vol. 77: 1993-1998).

In some embodiments of an affinity substrate according to the invention, the nucleic aptamers consist of DNA aptamers.

Consequently, in some embodiments of an affinity substrate according to the invention, said immobilized nucleic acids, where appropriate included in the structure of a compound of formula (I), consist of deoxyribonucleic acids.

In some other embodiments of an affinity substrate according to the invention, a first part of said nucleic acids consist of deoxyribonucleic acids, the remaining part consisting of ribonucleic acids.

Another advantage of the nucleic aptamers concerns the ease with which they can be produced, compared with the difficulties in synthesizing RNA aptamers, and also their cost price, which is significantly lower than the cost price of an RNA aptamer.

These specific embodiments are illustrated in the examples.

A subject of the present invention is also an affinity chromatography device for purifying a coagulation protein, comprising a container in which a suitable amount of an affinity substrate as defined in the present description is deposited.

Varied forms of containers for chromatography substrates are known in the prior art and are encompassed by the meaning of the term “container” above. The important characteristics of such a container encompass the presence of a means for feeding the affinity chromatography device with starting sample and of a means for output of the liquid after it has been brought into contact with the affinity substrate.

A subject of the present invention is also a method for immobilizing a coagulation protein on a support, comprising a step during which a sample containing said coagulation protein is brought into contact with an affinity substrate as defined above.

The expression “sample containing a coagulation protein” is intended to mean in general any type of liquid solution in which said coagulation protein is in suspension or is solubilized. Specific embodiments of such a sample, in particular in relation to the purification method described hereinafter, will be subsequently defined in the present description.

The present invention also relates to a method for purifying a coagulation protein, comprising the following steps:
a) bringing a sample containing a coagulation protein into contact with an affinity substrate as defined in the present description, in order to form complexes between (i) the nucleic aptamers immobilized on said affinity substrate and (ii) said coagulation protein, and
b) releasing the protein from the complexes formed in step a), and
c) recovering said coagulation protein in a purified form.

In some preferred embodiments, said sample contains a human coagulation protein. Advantageously, in these embodiments, the sample containing a coagulation protein of interest consists of a liquid sample which contains said protein of interest, including a liquid sample comprising said protein of interest and which is capable of also containing molecules of the homologous coagulation protein of a nonhuman mammal. In some embodiments of the purification method above, said sample consists of a biological solution, such as a body fluid, a cell, ground cell material, a tissue, ground tissue material, an organ or a whole organism.

In some embodiments of the purification method above, said sample consists of a liquid biological solution originating from an animal, such as blood, a blood derivative, mammalian milk or a mammalian milk derivative. Said sample can consist of plasma, plasma cryoprecipitate, clarified milk, or derivatives thereof.

In particularly preferred embodiments of the purification method above, said sample originates from an animal that is transgenic for the human protein of interest. Advantageously, the solution is milk from a mammal or a derivative of milk from a mammal that is transgenic for said human protein of interest. For the purpose of the invention, the transgenic animals encompass (i) nonhuman mammals such as cows, goats, rabbits, pigs, monkeys, rats or mice, (ii) birds or else (iii) insects such as mosquitoes, flies or silkworms. In some preferred embodiments, the animal that is transgenic for the human protein of interest is a nonhuman transgenic mammal, entirely preferably a doe rabbit that is transgenic for said human protein of interest. Advantageously, the transgenic mammal produces said recombinant human protein of interest in its mammary glands, owing to the insertion into its genome of an expression cassette comprising a nucleic acid encoding said protein of interest, which is placed under the control of a specific promoter allowing the expression of the transgenic protein in the milk of said transgenic mammal.

A method for producing said human coagulation protein in the milk of a transgenic animal can comprise the following steps: a DNA molecule comprising a gene encoding the protein of interest, said gene being under the control of a promoter of a protein naturally secreted in milk (such as the casein promoter, the beta-casein promoter, the lactalbumin promoter, the beta-lactoglobulin promoter or the WAP promoter), is integrated into an embryo of a nonhuman mammal. The embryo is then placed in a mammalian female of the same species. Once the mammal resulting from the embryo is sufficiently developed, lactation by the mammal is induced, and the milk is then collected. The milk then contains the recombinant human protein of interest.

An example of a method for preparing protein in the milk of a mammalian female other than a human being is given in document EP 0 527 063, the teaching of which can be reproduced for producing the protein of the invention. A plasmid containing the WAP (Whey Acidic Protein) promoter is produced by introducing a sequence comprising the promoter of the WAP gene, this plasmid being prepared in such a way as to be able to receive a foreign gene placed under the control of the WAP promoter. The plasmid containing the promoter and the gene encoding the protein of the invention is used to obtain transgenic doe rabbits by microinjection into the male pronucleus of embryos of doe rabbits. The embryos are then transferred into the oviduct of hormonally prepared females. The presence of the transgenes is revealed by the Southern technique using the DNA extracted from the young transgenic rabbits obtained. The concentrations in the milk of the animals are evaluated by means of specific radioimmunological assays.

Other documents describe methods for preparing proteins in the milk of a mammalian female other than a human being. Mention may be made, without being limited thereto, of the documents U.S. Pat. No. 7,045,676 (transgenic mouse) and EP 1 739 170 (production of von Willebrand factor in a transgenic mammal).

The purification method of the invention is also perfectly suitable for obtaining a purified coagulation protein from a sample of human blood plasma, or from a fraction of human blood plasma, for example the cryoprecipitated fraction of human blood plasma.

In some embodiments of the purification method above, the target coagulation protein is human.

In some embodiments of the purification method above, the sample comprises at least one nonhuman coagulation protein.

In some embodiments of the purification method above, said human coagulation protein is homologous to said nonhuman coagulation protein.

In some embodiments of the purification method above, said human coagulation protein is the homolog of said nonhuman coagulation protein.

In some embodiments of the purification method above, the starting sample can consist of the crude material, either the sample of human blood plasma, or a fraction thereof, or the body fluid of a nonhuman mammal that is transgenic for the protein of interest, and which contains said protein of interest to be purified. The body fluids of a transgenic nonhuman mammal encompass the milk or a fraction of the milk, for example a defatted fraction of the milk or alternatively a casein-micelle-depleted fraction.

However, the above embodiment is not the preferred embodiment of the purification method of the invention, in particular owing to the risk of clogging of the affinity substrate by the numerous proteins present in the crude starting sample.

In preferred embodiments, said starting sample consists of a liquid solution containing the coagulation protein of interest in suspension in said solution, said liquid solution consisting of an intermediate product generated during a multistep method for purifying a coagulation protein.

By way of illustration, for a method for purifying a coagulation protein from a body fluid of a nonhuman mammal that is transgenic for said protein, the starting sample may consist of an eluate of an ion exchange chromatography carried out using a filtrate of milk of said nonhuman transgenic mammal. This particular embodiment of a purification method according to the invention is illustrated in the examples.

In the same way, for a method for purifying the coagulation protein of interest from human plasma, the starting sample may consist of a filtrate of a deep filtration step carried out on the cryoprecipitated fraction of a sample of human plasma.

Generally, the conditions for using the affinity substrate in order to carry out the purification method of the invention are very similar to the customary conditions for using a conventional chromatography substrate, for example of the immunoaffinity substrate type on which ligand antibodies are immobilized. Those skilled in the art may, for example, refer to the book by Bailon et al. (Pascal Bailon, George K. Ehrlich, Wen-Jian Fung and Wolfgang Berthold, An Overview of Affinity Chromatography, Humana Press, 2000).

However, as will be detailed in the subsequent description, the conditions of elution step c) of the method of the invention are very advantageous for purifying a coagulation protein.

In step a), a suitable volume of the sample to be purified is brought into contact with the affinity substrate. Complexes are formed between (i) the nucleic aptamers immobilized on said affinity substrate and (ii) the coagulation protein of interest contained in the sample to be purified.

It has been shown in the examples that the conditions for capturing factor VII are improved when a buffer containing a low concentration of MgCl₂ or even a buffer free of MgCl₂ is used in step a).

The expression “buffer with a low concentration of MgCl₂” is intended to mean, according to the invention, a buffer of which the final MgCl₂ concentration is less than 1 mM.

A buffer of which the MgCl₂ concentration is less than 1 mM encompasses buffers of which the MgCl₂ concentration is less than 0.5 mM, 0.1 mM, 0.05 mM and 0.01 mM, advantageously equal to 0 mM.

In one particular embodiment, the method comprises a step a′) of washing the affinity substrate with a washing buffer. Advantageously, the method comprises a step a′) of washing the affinity substrate while increasing the ionic strength, i.e. with a washing buffer of which the ionic strength is increased compared with the ionic strength of step a). Advantageously, the ionic strength of the washing buffer is increased by 2- to 500-fold compared with the ionic strength of step a). Advantageously, the ionic strength of the washing buffer is increased by 100- to 500-fold, preferably by 200- to 500-fold, compared with the ionic strength of step a).

It has been shown in the examples that the use, in step a′), of a washing buffer having a high ionic strength, in particular a high NaCl concentration, makes it possible to effectively eliminate the substances bound nonspecifically to the affinity substrate without simultaneously affecting, in a detectable manner, the binding of factor VII to the affinity substrate.

In step a′), a washing buffer having a final NaCl concentration of at least 1 M is thus preferably used.

According to the invention, a washing buffer having a final NaCl concentration of at least 1 M encompasses washing buffers having a final NaCl concentration of at least 1.5 M, 2 M, 2.5 M or at least 3 M.

Preferably, a washing buffer used in step a′) of the method has a final NaCl concentration of at most 3.5 M. Advantageously, a washing buffer used in step a′) of the method has a final NaCl concentration of between 1.5 and 3.5, preferably between 2 and 3.5, preferably between 2.5 and 3.5, preferably between 3 and 3.5.

It has also been shown in the examples that the use, in step a′), of a washing buffer having a high hydrophobicity, in particular a high propylene glycol concentration, makes it possible to effectively eliminate the substances bound nonspecifically to the affinity substrate without simultaneously affecting, in a detectable manner, the binding of factor VII to the affinity substrate.

In step a′), a washing buffer having a final propylene glycol content of at least 20% (v/v) is thus preferably used.

According to the invention, a washing buffer having a final propylene glycol content of at least 20% encompasses washing buffers having a final propylene glycol content of at least 25%, 30%, 35%, 40%, 45%, 50%, 55%, or at least 60% by volume, relative to the total volume of the washing buffer.

Preferably, a washing buffer used in step a′) of the method has a final propylene glycol content of at most 50%. Advantageously, a washing buffer used in step a′) of the method has a final propylene glycol content of between 20% and 50%, preferably between 30% and 50%.

According to one particular embodiment, the washing buffer used in step a′) contains both NaCl and propylene glycol as described above.

Step b) consists of a step of eluting the molecules of the coagulation protein of interest having formed complexes with the nucleic aptamers during step a).

As is illustrated in the examples, a specific advantage of the purification method above is the possibility of carrying out the elution step by bringing the complexes formed between (i) the nucleic aptamers immobilized on said affinity substrate and (ii) said coagulation protein into contact with a divalent-ion-chelating agent, such as EDTA.

This technical advantage, which is made possible by virtue of the characteristics of the affinity substrate of the invention, allows the elution of the coagulation protein without requiring any recourse to the use of drastic elution conditions, capable of at least partially denaturing the coagulation protein of interest. Said drastic conditions which are avoided encompass the use of an acidic pH for the elution step, which is commonly implemented for methods for purifying proteins on affinity substrates that are known, and most particularly on affinity substrates comprising immobilized antibodies.

Thus, in some embodiments of the purification method above, step b) is carried out by bringing the affinity substrate into contact with an elution buffer containing a divalent-ion-chelating agent, preferably EDTA.

By way of illustration, the elution buffer may contain a final EDTA concentration of at least 1 mM and of at most 30 mM.

The expression “at least 1 mM” encompasses at least 2, 3, 4, 5, 6, 7, 8, 9 or 10 mM.

The expression “at most 30 mM” encompasses at most 29, 28, 27, 26, 25, 24, 23, 22, 21, 20, 19, 18, 17, 16, 15, 14, 13, 12 or 11 mM.

In step c), the purified coagulation protein of interest is recovered by collecting the eluate liquid obtained at the end of step b).

At the end of step c), a purified liquid composition of the coagulation protein of interest is obtained. Said purified liquid composition can then be treated appropriately, according to any known technique for conditioning and storing proteins, including by direct bottling or bottling after dilution with a suitable solution, or else by freeze-drying, preferentially under sterile and apyrogenic conditions, and then storage under appropriate conditions, at ambient temperature, at −4° C. or else at a low temperature, depending on the type of conditioning selected.

As has already been mentioned previously in the present description, the affinity substrate of the invention can, with the successive cycles of use for purifying a coagulation protein of interest, experience a reduction in its absorption capacity, for example owing to the fact that elution step c) does not make it possible to systematically release all of the molecules of coagulation protein, thereby reducing the number of free aptamer sites for the subsequent purification cycles.

As for all known chromatography substrates, it is therefore necessary, at appropriate times, to carry out a step of regenerating the affinity substrate, in order to release all of the molecules of coagulant protein from said substrate, and to eliminate any substance that may be bound to the solid material of the affinity substrate, generally by nonspecific binding.

Thus, in some embodiments, the purification method of the invention comprises an additional step d) of regenerating the affinity substrate by bringing said affinity substrate into contact with a regenerating solution.

Varied buffers for regenerating chromatography substrates, in particular affinity chromatography substrates, are well known by those skilled in the art, and can be used in step d) of the method. Those skilled in the art may refer, for example, to the book by Mohr et al. (Affinity Chromatography: Practical and Theoretical Aspects, Peter Mohr, Klaus Pommerening, Edition: illustrated, CRC Press, 1985).

By way of illustration, step d) of regenerating the affinity substrate can be carried out by bringing said substrate into contact with a 50 mM Tris, 50% ethylene glycol buffer solution as is illustrated in the examples.

As is illustrated in the examples, the purification method above makes it possible to obtain a coagulation protein at a very high degree of purity, optionally at a degree of purity of greater than 99.95% by weight, relative to the total weight of the proteins contained in the purified final product.

Another advantage of the purification method above, in particular in the embodiments in which the starting sample consists of a sample comprising the human coagulation protein of interest in recombinant form as a mixture with proteins naturally produced by the nonhuman transgenic mammal, is that the final composition comprising the recombinant human protein of interest at a high degree of purity is substantially free of proteins originating from said transgenic mammal, and in particular substantially free of proteins of said mammal, which are homologs of said recombinant human protein.

By way of illustration, it has been shown in the examples that recombinant human factor VII produced in the milk of a transgenic rabbit, then purified with the purification method defined in the present description, comprises less than 1.5% by weight of the proteins of said transgenic mammal, relative to the weight of said proteins initially contained in the starting sample. In the same implementation of the purification method according to the invention, 85% by weight of the recombinant human factor VII present in the starting sample was contained in the final product at a high degree of purity with respect to recombinant human factor VII, greater than 99.95% of the weight of the proteins present.

A subject of the present invention is also a purified composition of a recombinant human coagulation protein comprising at least 99.9% by weight of said recombinant human protein and which is substantially free of nonhuman proteins.

The present invention also relates to a purified composition of a recombinant human coagulation protein comprising at least 99.9% by weight of said recombinant human protein and at most 0.1% by weight of nonhuman proteins, the percentages by weight being expressed relative to the total weight of proteins of said purified composition.

In the purified composition above, “at least 99.9%” encompasses at least 99.91%, 99.92%, 99.93%, 99.94%, 99.95%, 99.96%, 99.97%, 99.98% and 99.99%. In the purified composition above, “at most 0.1%” encompasses at most 0.09%, 0.08%, 0.07%, 0.06%, 0.05%, 0.04%, 0.03%, 0.02% and 0.01%.

The present invention also relates to a purified composition as defined above, that can be used as a medicament.

The invention further relates to a pharmaceutical composition comprising a purified composition of a recombinant human coagulation protein as defined above, in combination with one or more pharmaceutically acceptable excipients.

The invention also relates to a purified composition as defined above, for treating coagulation disorders.

The invention also relates to the use of a purified composition as defined above, for producing a medicament for treating coagulation disorders.

Specific embodiments of aptamers which bind specifically to a coagulation protein, which can be advantageously used according to the invention, are described below.

### Specific Embodiments of Aptamers that can be Used According to the Invention

The applicant has constructed a family of nucleic aptamers which bind specifically to coagulation proteins, and in particular to human factor VII/VIIa, for which it has been able to show the existence of relationships between (i) the common structural characteristics and (ii) the common functional characteristic(s).

From a structural point of view, the family of nucleic acids, or nucleic aptamers, which bind specifically to human factor VII/VIIa and which can be used according to the invention comprises at least 15 consecutive nucleotides of a polynucleotide having at least 40% nucleotide identity with the nucleic acid of formula (I) below:

5′-[SEQ ID No.1]x-[SEQ ID No. X]-[SEQ ID No.2]y-3′ (I),

in which:
“SEQ ID No. X” consists of a nucleic acid chosen from the group consisting of the nucleic acids of sequences SEQ ID No. 3 to SEQ ID No. 85 and SEQ ID No. 87 to SEQ ID No. 100,
“x” is an integer equal to 0 or 1, and
“y” is an integer equal to 0 or 1.

In some embodiments, the acid of sequence SEQ ID No. X has a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

In other embodiments, the nucleic acid of sequence SEQ ID No. X has a length of 43, 44, 45, 46, 47, 48 or 49 nucleotides.

In some other preferred embodiments, the nucleic acid of sequence SEQ ID No. X has a length of 43, 44 or 45 nucleotides.

As already mentioned previously, the nucleic acid of formula (I) is at least 15 nucleotides in length.

In some embodiments, the nucleic acid of formula (I) is at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or 81 nucleotides in length, which encompasses the nucleic acids having exactly each of the specified lengths.

In some embodiments of the method for obtaining the aptamers of formula (I), the successive selection cycles carried out in order to construct the family of nucleic acids of interest which bind specifically to coagulation proteins have resulted in isolating and characterizing, at each successive selection step, sets and subsets of nucleic aptamers comprising, at their 5′ and 3′ ends respectively, the sequences SEQ ID No. 1 and SEQ ID No. 2, structurally framing a variable sequence SEQ ID No. X. In the main family of nucleic aptamers of the invention, all the variable sequences SEQ ID No. X have, between them, a nucleotide sequence identity of at least 40%. This means that, for the sequence SEQ ID No. X, the structural constraints, for retaining the property of binding to coagulation proteins, are much less than the structural constraints for the sequences located, respectively, at the 5′ and 3′ ends of these nucleic aptamers.

When the integer “x” is equal to 0 and the integer “y” is equal to 1, the nucleic aptamers of the invention encompass the nucleic acids comprising at least 15 consecutive nucleotides of a polynucleotide having at least 40% nucleotide identity with the nucleic acid of formula (1-1) below:

5′-[SEQ ID No. X]-[SEQ ID No. 2]-3′ (I-1).

When the integer “x” is equal to 1 and the integer “y” is equal to 0, the nucleic aptamers of the invention encompass the nucleic acids comprising at least 15 consecutive nucleotides of a polynucleotide having at least 40% nucleotide identity with the nucleic acid of formula (I-2) below:

5′-[SEQ ID No. 1]-[SEQ ID No. X]-3′ (I-2).

When the integer “x” is equal to 0 and the integer “y” is equal to 0, the nucleic aptamers of the invention encompass the nucleic acids comprising at least 15 consecutive nucleotides of a polynucleotide having at least 40% nucleotide identity with the nucleic acid of formula (I-3) below:

5′-[SEQ ID No. X]-3′ (I-3).

The nucleic aptamers above therefore encompass the nucleic acids comprising at least 15 consecutive nucleotides of a polynucleotide having at least 40% nucleotide identity with a nucleic acid chosen from the group consisting of the nucleic acids of sequences SEQ ID No. 3 to SEQ ID No. 85 and SEQ ID No. 87 to SEQ ID No. 100.

Generally, a first polynucleotide having at least 40% nucleotide identity with a second polynucleotide or a nucleic acid has at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 100% nucleotide identity with said second polynucleotide or nucleic acid.

In some embodiments of a nucleic acid of the invention comprising the sequence SEQ ID No. X, said sequence SEQ ID No. X is chosen from the group consisting of the nucleic acids having at least 15 consecutive nucleotides of a sequence having at least 40% nucleotide identity with at least one of the sequences SEQ ID No. 3 to SEQ ID No. 85 and SEQ ID No. 87 to SEQ ID No. 100.

In some embodiments of a nucleic acid of the invention comprising the sequence SEQ ID No. X, said sequence SEQ ID No. X is chosen from the group consisting of the nucleic acids having at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 100% nucleotide identity with at least one of the sequences SEQ ID No. 3 to SEQ ID No. 85 and SEQ ID No. 87 to SEQ ID No. 100.

It results from the aforementioned that the present invention encompasses a family of single-stranded nucleic acids having at least 15 consecutive nucleotides of the series of formula (I) defined above.

The “percentage identity” between two nucleic acid sequences, for the purpose of the present invention, is determined by comparing the two sequences optimally aligned, through a window of comparison.

The part of the nucleotide sequence in the window of comparison can thus comprise additions or deletions (for example “gaps”) compared with the reference sequence (which does not comprise these additions or these deletions) in such a way as to obtain an optimum alignment between the two sequences.

The percentage identity is calculated by determining the number of positions at which an identical nucleic base is observed for the two compared sequences, and then by dividing the number of positions at which there is identity between the two nucleic bases by the total number of positions in the window of comparison, and then by multiplying the result by one hundred in order to obtain the percentage nucleotide identity of the two sequences with respect to one another.

The optimal alignment of the sequences for the comparison can be carried out by computer using known algorithms.

Entirely preferably, the percentage sequence identity is determined by means of the CLUSTAL W software (version 1.82), the parameters being fixed as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GRAP DISTANCES=“hide”.

The present invention is also illustrated by the following examples.

### EXAMPLES

### Example 1

### Preparation of an Affinity Substrate

The affinity substrate was prepared from a solid substrate material consisting of a matrix onto which streptavidin (streptavidin-agarose—Novagen®) was grafted.

A volume of 1 ml of gel was placed in a container consisting of a column (i.d. 11 mm). The gel was washed with purified water, in order to remove the storage solvent.

### The Characteristics of the Gel are:

Biotin adsorption capacity: 85 nanomol/ml of gel

Functional test: capture >99% of biotinylated thrombin over the course of 30 minutes at AT

Other tests: Protease-free, endo/exonuclease-free, RNase-free

Preservative: 100 mM sodium phosphate pH 7.5+NaN₃ 0.02

The outlet of the packed column (gel bed height=1 cm) is connected to an absorbance detector equipped with a UV filter at 254 nm and a recording device.

The biotinylated anti-human FVII nucleic aptamers of sequence SEQ ID No. 86 are solubilized in purified water at a final concentration of 0.5 mg/0.187 ml, i.e. a final molar concentration of 0.1 mM. The solution of nucleic aptamers was activated at 95° C. according to the standard cycle, for the immobilization of the aptamers on the solid substrate material.

The solution of nucleic aptamers was prediluted with 4.8 ml of purified water and then 1.5 ml of Me⁺⁺ buffer (5× concentrated).

The absorbance detector is adjusted to 1 AUFS (absorbance unit full scale) and the OD at 254 nm of this solution is recorded at 0.575 AU₂₅₄.

The solution of biotinylated nucleic aptamers is injected onto the prepacked streptavidin-agarose gel and recirculated with a peristaltic pump at a flow rate of 2.5 ml/minute, i.e. a contact time on the gel of 24 seconds (inlet/outlet I/O). Under these conditions, the OD at 254 nm stabilizes rapidly at 0.05 AU₂₅₄, i.e. a theoretical coupling value of 91%, i.e. 0.455 mg of nucleic aptamers per milliliter of gel.

Washing with a 10 mM CaCl₂+4 mM MgCl₂ buffer and then in 2 M NaCl is carried out in order to eliminate the nucleic aptamers which are not bound specifically to the streptavidin molecules grafted onto the solid substrate material.

### Example 2

### Method for Purifying Recombinant Human Factor VII

The aptamer affinity substrates were tested using a purified preparation of FVII/FVIIa prepared according to the technique described in PCT application No. WO2008/099077.

### Preparation of the Sample to be Purified

The starting biological material is transgenic rabbit milk containing recombinant human FVII. The expression cassette comprises the human FVII transgene placed under the control of the γ-casein gene promoter.

Briefly, 140 milliliters of milk were collected from 2 rabbits in first lactation between day 4 and day 12 after having given birth.

The average titer of amidolytic FVII (biologically activatable FVII) in the milk collected is 928 IU/ml. The milks are stored at a temperature of −80° C.

For the test, the rabbit milks are thawed in a water bath at a temperature of 37° C., and are then diluted with a sodium citrate solution to give a final citrate concentration of 62 g/l at a pH of 7.5.

The treatment with sodium citrate makes it possible to destabilize the phosphocalcic casein micelles.

The lipid-rich protein solution of milk is then clarified over a sequence of filters, respectively (i) depth filter of 15 to 0.5 μm porosity threshold and then (ii) membrane filter at 0.2 μm.

A volume of 360 ml of filtered solution having an FVII titer of 198 IU/ml, i.e. 36 mg of transgenic FVII, is prepurified on an MEP-HyperCel® chromatography gel (Pall BioSepra) having a volume of 16 ml. This capture gel makes it possible to eliminate 95% of the milk proteins, including the majority of caseins, while at the same time retaining 60% of the initial amount of FVII.

An amount of 17.5 mg of low-purity FVII (˜5%) obtained at the end of the above step is purified by ion exchange chromatography using a Q-sepharose® XL gel (GE Healthcare) having a volume of 20 ml, the human FVII being eluted with a volume of 78 ml of buffer comprising 5 mM of calcium chloride. The concentration of amidolytic FVII is 337 IU/ml, i.e. 0.17 mg of FVII/ml, and the concentration of total proteins is estimated at 0.18 mg/ml by measurement of OD at 280 nm and ε^{1%}=13, i.e. an FVII purity of 94%.

The residual proteins originating from the rabbit milk are difficult to separate from the FVII at this stage, either because there are structural homologies, such as GLA-domain or EGF-domain proteins, or else because there are physicochemical homologies (similar ionic charge and/or molecular size). Conventional techniques allow an improvement in purity up to 99.95% by means of orthogonal techniques (combination of separation on hydroxyapatite gel and by size exclusion chromatography). However, for repeated injection in humans, the load with respect to exogenous proteins accepted for genetic recombination proteins must not exceed 50 ppm, i.e. a purity >99.995%. Such a purity appears to be attainable only after purification on an affinity matrix.

### Step of Purifying Recombinant Human FVII on the Affinity Substrate of the Invention

A volume of 6 ml of the solution of purified human FVII (1.1 mg of FVII) obtained at the end of the preceding step is used for the step for purifying the recombinant human FVII at a high level of purity with the affinity substrate of the invention.

The FVII solution obtained in the preceding step, preadjusted to 4 mM MgCl₂ and 10 mM CaCl₂ and pH 7.5, is injected onto the aptamer-agarose gel (affinity substrate) with a peristaltic pump at a flow rate of 0.1 ml/minute, i.e. a contact time with the affinity substrate of 10 minutes (I/O).

After injection, the gel is washed in 50 mM tris+NaCl 50 mM+4 mM MgCl₂+10 mM CaCl₂ buffer at pH 7.5.

A nonadsorbed volume of 10 ml of solution is collected.

The FVII is eluted with a 50 mM tris+EDTA 10 mM buffer at pH 7.5. The collection of the elution peak is carried out according to the OD profile.

According to the molar calculations, the amount of nucleic aptamers immobilized in the affinity substrate is 17 nanomol, which corresponds, for a mole-for-mole interaction with the FVII molecules, to an absolute capacity of the affinity substrate of 0.9 mg of FVII.

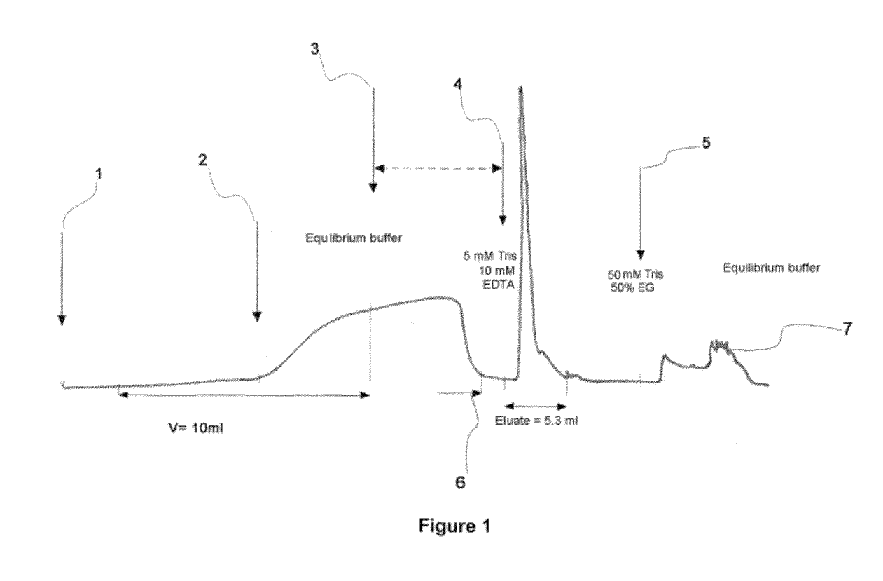

FIG. 1 illustrates a chromatography profile of the recombinant human FVII produced in the rabbit milk, with continuous monitoring of the absorbance (OD) values at 254 nanometers.

In FIG. 1, the inflexion (2) of the absorption curve, after the moment of the injection (1), illustrates the beginning of the saturation of the affinity substrate with the recombinant human FVII. At time (3), the injection of recombinant human FVII is stopped. To illustrate the linear scale of the times in FIG. 1, it is indicated that the duration between the injection start time (1) and the injection end time (2) is 10 minutes. The affinity substrate continues to be saturated with the coagulation protein of interest: complexes between (i) the anti-FVII nucleic aptamers of the affinity substrate and (ii) the molecules of recombinant human FVII initially contained in the composition to be purified have been formed. After the composition to be purified has been passed over the column, a step of washing (6) the column with the washing buffer specified above is carried out. The elution step is then carried out, by injection, at time (4), of the buffer solution comprising a final EDTA concentration of 10 mM. The absorption peak illustrates the release of the recombinant human FVII from the nucleic aptamer/recombinant FVII complexes. It is noted that the molecules of recombinant human FVII are released rapidly and therefore in a small volume. Consequently, by virtue of the affinity substrate of the invention, an elution solution with a high concentration of recombinant human FVII protein is obtained. At time (5), a step of regenerating the affinity substrate is carried out with a 50 mM tris buffer. The absorbance peak visible at (7) corresponds to the substances released from the affinity substrate owing to the regeneration step.

### Dynamic Binding Capacity of the Affinity Substrate

Table 1 below gives the results of the test, which show a dynamic binding capacity of 0.45 to 0.49 mg/ml of the affinity matrices, i.e. 50 to 55% of bioavailable ligands.

In EDTA, a dynamic elution of approximately 75% is calculated.

TABLE 1
recombinant human FVII and total protein results of the
aptamer-agarose matrix tests:

| | Recombinant | | Proteins | | DBC | |
|---|---|---|---|---|---|---|
| | FVII | | (total mg) | | (mg/ml) | DE (%) |
| Start | 2228 | 100% | 1.42 | 100% | | |
| Final | | | | | | |
| Nonadsorbed | 924 | 41% | 0.57 | 40% | | |
| Eluate | 971 | 44% | 0.61 | 43% | 0.49 | 74% |
| Results by | | 85% | | 83% | | |
| weight | | | | | | |

Start: starting sample
Final: fraction composition
DBC: dynamic binding capacity
DE: dynamic elution; which represents the ratio between the eluted recombinant FVII and the adsorbed recombinant FVII, expressed as a percentage

### Specific Separation Capacity of the Affinity Substrate

The affinity substrates were evaluated in terms of specificity by means of an ELISA assay specific for rabbit milk proteins.

The results are represented in table 2 below.

TABLE 2
affinity substrate specificity results:

| | Recombinant FVII | | Rabbit milk | | RMP | % FVII |
|---|---|---|---|---|---|---|
| | (total mg) | | proteins (RMP) | | (ppm) | purity |
| Start | 1.11 | 100% | 16992 | 100% | 16782 | 98.32% |
| Final | | | | | | |
| Nonadsorbed | 0.46 | 41% | 14590 | 40% | 34738 | 96.53% |
| Eluate | 0.49 | 44% | 217 | 43% | 492 | 99.95% |
| Results by | | 85% | | 83% | | |
| weight | | | | | | |

The results in table 2 above show that an average of 2 log₁₀ of elimination by the aptamers-agarose is obtained, taking the purity of the transgenic human FVII from 98.3% to 99.95%. This shows a good specificity of the aptamers with respect to human FVII and very few interactions with the residual rabbit milk proteins.

An improvement is possible by means of intermediate washes, before elution, with solutions such as 2M NaCl and/or propylene glycol or ethylene glycol at 50% if, under these conditions, the FVII is not eluted.

The results of example 2 illustrate the excellent characteristics of the affinity substrates on aptamer-agarose gel (Apta-2) with a dynamic binding capacity of at least 1 mg of FVII per mg of ligand with an elution yield of at least 75%. The specificity is also well established with a clear improvement in purity (˜99.95%), with an elimination of 2 log₁₀ of the residual rabbit milk proteins RMP. The final level comes to approximately 500 ppm over these 2 nonoptimized tests.

### Example 3

### Method for Purifying Human Plasma Factor IX

### A. Materials and Methods

### A.1. Affinity Chromatography Substrate

Mapt-1 affinity gel material, without spacer, theoretical ligand density 0.46 mg/ml: volume 1 ml.
The aptamer is directly bonded to the chromatography substrate material by means of a chemical coupling reaction.
The aptamer used is the aptamer of sequence SEQ ID No. 101.

### A.2. Starting Product

The starting product consists of a composition enriched in factor IX derived from human plasma, which is sold by LFB under the name Betafact®.
Starting material injected: Betafact MPVP (plasma FIX at 60% purity), load of 200 IU (i.e. 800 μg) of factor IX per ml of gel, contact time 10 minutes.

A.3. Purification Protocol

Gel equilibration: 0.050 M Tris-HCl, 0.010 M CaCl₂, pH 7.5,

### Elution: 0.020 M Tris-HCl, 0.010 M EDTA, pH 7.5,

Regeneration: 0.020 M Tris-HCl, 1 M NaCl, 50% propylene glycol, pH 7.5.
The protein peaks are detected by measuring the absorbance value at the wavelength of 280 nanometers.

### A.4. Protocol for Analysis by Electrophoresis on SDS PAGE Gel

10-well NOVEX gels (Invitrogen), 4-12%, Bis-Tris; MES running buffer, migration at 200 V for 50 min. CBB (G250) or AgNO₃ (GE kit) staining.

### B. Results

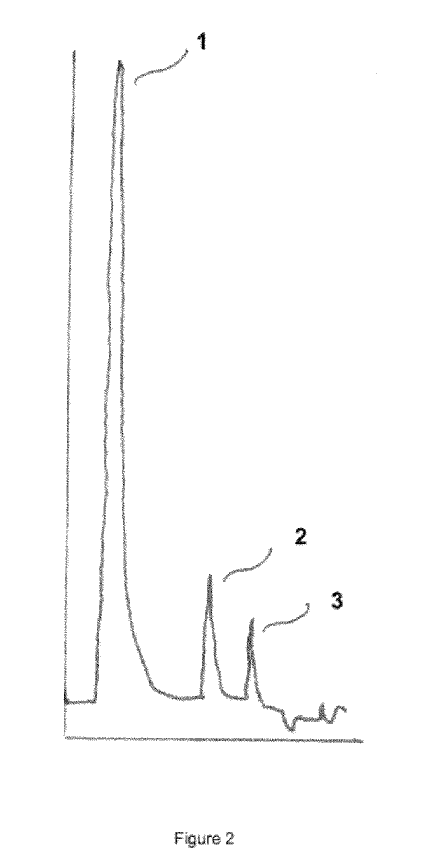

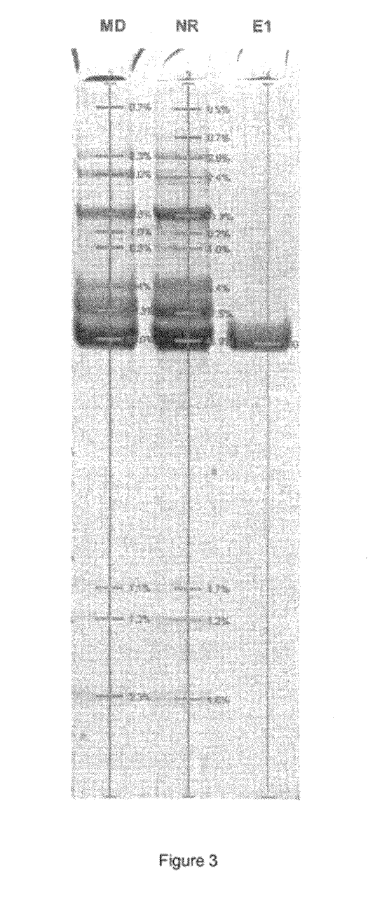

The results are illustrated in FIGS. 2 and 3.

FIG. 2 represents the curve of the values of the measurement of absorbance at 280 nm as a function of time. In FIG. 2, peak No. 1 corresponds to the fraction of the starting product which was not retained on the column. Peak No. 2 corresponds to the elution fraction. Peak No. 3 corresponds to the fraction desorbed from the substrate by carrying out the regeneration step.

FIG. 3 is an image of an SDS PAGE electrophoresis gel. From left to right of the gel in FIG. 3, the lanes represent the results of migration of the following starting products:
lane “MD”: the Betafact® starting composition,
lane “NR”: the nonretained fraction corresponding to peak No. 1 of the chromatographic profile of FIG. 2,
lane “E1”: the elution fraction corresponding to peak No. 2 of the chromatographic profile of FIG. 2.

TABLE 3

| Stage | % FIX |
|---|---|
| Starting product | 51% |
| Nonretained | 44% |
| Eluate | 100% |

Table 3 recapitulates the percentages of purity of the FIX obtained by integration of the electrophoretic profile in the various fractions. The % FIX is calculated according to a procedure well known to those skilled in the art, by quantitative integration of the densities of the electrophoretic bands of the gel stained with CBB (numerical data corresponding to FIG. 3). The quantitative integration of the densities of the electrophoretic bands can be obtained by scanning the gel with a suitable scanner.

The results represented in FIG. 3 and table 3 confirm those of FIG. 2. These results illustrate that the chromatography substrate on which the aptamer is immobilized allows the specific purification of human factor IX from a complex medium such as a plasma fraction enriched in factor IX.

It can be concluded that the eluate exhibits a good electrophoretic purity and is characterized by the fact that the functionality of the FIX is maintained. This experiment shows the capacity of the aptamer of sequence SEQ ID No. 101 directly coupled to the chromatographic substrate, and therefore in the absence of a spacer chain, to bind and to purify FIX in a complex medium containing plasma impurities.

### Example 4

### Method for Purifying Recombinant Factor IX Contained in an Extract of Transgenic Sow's Milk

### A. Materials and Methods

### A.1. Affinity Chromatography Substrate

Affinity gel material on which the Mapt-1 aptamer is immobilized by direct coupling without a spacer chain. Theoretical ligand density 0.46 mg/ml: volume 1 ml.
The aptamer is directly bonded to the chromatography substrate material by means of a chemical coupling reaction.
The aptamer used is the Mapt 1 aptamer of sequence SEQ ID No. 101.

### A.2. Starting Product

The starting product consists of a transgenic sows' milk clarified and prepurified on MEP HyperCel: 1.8% purity. Sample dialyzed against the buffer for adsorption/equilibration of the resin in order to remove the sodium citrate. Load of 302 IU (i.e. 1200 μg) of factor IX per ml.

### A.3. Purification Protocol

Gel equilibration: 0.050 M Tris-HCl, 0.010 M CaCl₂, pH 7.5,

### Elution: 0.020 M Tris-HCl, 0.010 M EDTA, pH 7.5,

Regeneration: 0.020 M Tris-HCl, 1 M NaCl, 50% propylene glycol, pH 7.5.
The sample is injected with a flow rate of 0.1 ml/min for 10 min, the gel is then washed for 5 min at 0.5 ml/min. The elution and the regeneration are carried out by injection of 2 ml of each of the buffers with a flow rate of 0.5 ml/min.
The protein peaks are detected by measuring the value of absorbance at the wavelength of 280 nanometers.

### A.4. Protocol for Analysis by SDS PAGE Gel Electrophoresis

10-well NOVEX gels (Invitrogen), 4-12%, Bis-Tris; MES running buffer, migration at 200V for 50 min. CBB (G250) or AgNO₃ (GE kit) staining.
A.5. Protocol for Measuring the Specific Activity with Respect to Factor IX
The measurement of the amount of FIX (antigen measurement) was carried out with the Serachrom 0943 FIX Ag kit (Stago) according to the supplier's recommendations.
The measurement of the enzymatic activity of the FIX was carried out by means of a chromogenic test with the Biophen FIX kit, ref. 221802 (Hyphen BioMed) according to the supplier's recommendations.
The specific activity is calculated according to the following ratio:
Enzymatic activity of the FIX/amount of FIX.

### B. Results

The results are illustrated in FIGS. 4 and 5.
FIG. 4 represents the curve of the values of the measurement of absorbance at 280 nm as a function of time. In FIG. 4, peak No. 1 corresponds to the fraction of the starting product which was not retained on the column. Peak No. 2 corresponds to the elution fraction. Peak No. 3 corresponds to the fraction desorbed from the substrate by carrying out the regeneration step.
The results in FIG. 4 show that the elution peak is very narrow, which illustrates the very high specificity for human factor IX of the chromatography substrate on which the aptamer is immobilized.
FIG. 5 is an image of an SDS PAGE electrophoresis gel. From left to right of the gel in FIG. 5, the lanes represent the results of migration of the following starting products:
lane “E5”: the starting composition of transgenic sow's milk containing transgenic human factor IX prepurified by an MEP HyperCel chromatographic step,
lane “E6”: the nonretained fraction corresponding to peak No. 1 of the chromatographic profile of FIG. 4,
lane “E7”: the elution fraction corresponding to peak No. 2 of the chromatographic profile of FIG. 4,
lane “E8”: the elution fraction collected downstream of peak No. 2 and upstream of peak No. 3 of the chromatographic profile of FIG. 4,
lane “T FIX”: purified factor IX control.
   The results represented in FIG. 5 confirm those of FIG. 4. These results illustrate that the chromatography substrate on which the aptamer is immobilized allows the specific purification of human factor IX from a complex medium such as a plasma fraction enriched in factor IX.
   Furthermore, the results of the example illustrate the high degree of enrichment in human factor IX which is obtained after passing the starting product of complex composition over the affinity chromatography substrate on which the Mapt 2 aptamer of sequence SEQ ID No. 86 is immobilized.
   It can be concluded that the eluate exhibits a good electrophoretic purity with a considerable gain in purity compared with the starting product (>26-fold). The second band identified in the eluate certainly corresponds to another form of FIX.

### Example 5

### Method for Purifying Human Plasma Factor Vii

### A. Materials and Methods

### A.1. Affinity Chromatography Substrate

Affinity gel material on which was immobilized the “Mapt-2 core” aptamer coupled directly to biotin, without a spacer chain between the aptamer and the biotin. The aptamer is immobilized on a streptavidin gel (supplier Novagen) by means of a 5′-terminal biotin, with a theoretical ligand density of 0.4 mg/ml: volume 1 ml.
The aptamer used is the Mapt-2 core aptamer of sequence SEQ ID No. 20.

### A.2. Starting Product

The starting product consists of a composition of human plasma factor VII purified to 98%.

### A.3. Purification Protocol

Gel equilibration: 0.050 M Tris-HCl, 0.010 M CaCl₂, 0.05 mM MgCl₂, pH 7.5,

### Elution: 0.020 M Tris-HCl, 0.010 M EDTA, pH 7.5.

240 μg of human plasma factor VII purified to 98% is injected with a flow rate of 0.5 ml/min in equilibration buffer.
After detection of the peak of nonretained material, 2 column volumes of elution buffer are injected.
The protein peaks are detected by measuring the value of absorbance at the wavelength of 280 nanometers.

### B. Results

The results are illustrated in FIGS. 6 and 7.
FIG. 6 represents the curve of the values of the measurement of absorbance at 280 nm as a function of time. In FIG. 6, peak No. 1 corresponds to the fraction of the starting product which was not retained on the column. Peak No. 2 corresponds to the elution fraction.

The starting product and also the eluted product were analyzed by SDS PAGE with silver nitrate staining in order to visualize the elimination of the impurities. FIG. 7 represents this gel: lane No. 1 corresponds to the fraction of the starting product and lane No. 2 to the elution fraction. Despite the considerable purity of the starting product, it is noted that the eluted fraction no longer contains contaminants or degraded forms.

The results of FIGS. 6 and 7 show that the aptamer of sequence SEQ ID No. 20 is capable of binding human FVII and of specifically eluting it in the presence of EDTA.

### Example 6

### Absence of Binding of the Aptamer to Rabbit FVII

### A. Materials and Methods

### A.1. Affinity Chromatography Substrate

Affinity gel coupled to streptavidin, on which the aptamer of sequence SEQ ID No. 86 was immobilized by means of a spacer chain (supplier Novagen) via a 5′-terminal biotin, with a theoretical ligand density of 0.35 mg/ml: volume 1 ml.
The aptamer used is the aptamer of sequence SEQ ID No. 86

### A.2. Starting Product

Eluate of hydroxyapatite enriched in rabbit FVII obtained by purification from rabbit plasma, contact time 10 minutes, flow rate 0.5 ml/min.

### A.3. Purification Protocol

Gel equilibration: 0.050 M Tris-HCl, 0.010 M CaCl₂, 0.05 mM MgSO₃, pH 7.5,
Elution: 0.050 M Tris-HCl, 0.010 M EDTA, pH 7.5, 36 μg are injected into the gel with a contact time of 10 minutes. The elution is carried out by injecting 2 ml of elution buffer.
The protein peaks are detected by measuring the value of absorbance at the wavelength of 280 nanometers.

### A.4. Protocols for Analysis of the Fractions in Terms of Proteins and in Terms of Factor VII

The fractions are analyzed for their amidolytic activity by chromogenic assay using a Stago kit according to the supplier's recommendations (factor Vila StatClot kit). The amidolytic activity is then converted to μg of FVII contained in said fraction.

### B. Results

The results are illustrated in table 4 below.

TABLE 4

| | | | | | | Step |
|---|---|---|---|---|---|---|
| | Proteins | FVIIam | Volume | Amount | Purity | yield |
| Steps | (mg/ml) | (IU/ml) | (ml) | FVII (μg) | (%) | (%) |
| Starting | 1.38 | 57 | 2.5 | 71 | 2% | 100% |
| material | | | | | | |
| Dialyzed | 0.97 | 21.8 | 3.4 | 36.7 | 1% | 52% |
| eluate | | | | | | |
| Mapt-2 eluate | NA | 0.04 | 4.0 | 0.08 | NA | 0.2% |

The results in table 4 show that the rabbit factor VII is not retained on the affinity gel on which the Mapt-2 aptamer is immobilized.

### Example 7

### Specific Embodiments of a Protocol for Interaction of Human Factor VII with an Aptamer on Biacore (Resistance to NaCl)

A. Materials and Methods

A solid substrate on which molecules of the nucleic aptamer of the invention of sequence SEQ ID No. 86, also denoted herein “Mapt2”, were immobilized was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 3772 RU (1 RU corresponds approximately to 1 μg of immobilized product per mm²).

Purified transgenic human FVII obtained from transgenic rabbit milk (FVII HPTG, purity: 98%) was diluted in running buffer (50 mM Tris, 50 mM NaCl, 10 mM CaCl₂, 4 mM MgCl₂, pH 7.4) so as to obtain a sample having an FVII concentration of 200 mM.

The sample was injected onto the chip (solid substrate) containing the Mapt2 aptamer immobilized by means of a biotin-streptavidin interaction. Next, buffers containing an increasing concentration of NaCl were injected onto the solid substrate (3 series of injections ranging from 1 M NaCl to 3 M NaCl). All the injections were carried out with a flow rate of 30 μl/min for 60 sec after the injection. After the 3 series of injections with the 3 buffers containing NaCl, elution buffer (10 mM EDTA) was then injected for 75 sec with a flow rate of 30 μl/min in order to detach the FVII HPTG from the aptamer.

These analyses are carried out with the RPS Biacore T100 apparatus (GE). The modeling of the interactions recorded is carried out by means of the Biaevaluation software (GE).

The curves of binding of the Mapt2 immobilized aptamer to the transgenic human FVII were calculated with the dedicated module of the Biacore® control software, version 1.2.

The results of binding of the Mapt2 aptamer to human FVII made it possible to determine that the binding of the Mapt2 aptamer to human FVII is not detrimentally modified by the injection of the buffers containing NaCl.

### B. Results

The results are illustrated in FIG. 8.

### Example 8

### Specific Embodiments of a Protocol for Interaction of Human Factor VII with an Aptamer on Biacore (Resistance to Propylene Glycol)

A. Materials and Methods

A solid substrate on which molecules of the nucleic aptamer of the invention of sequence SEQ ID No. 86, also denoted herein “Mapt2”, were immobilized was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 5319 RU (1 RU corresponds approximately to 1 μg of immobilized product per mm²).

Purified transgenic human FVII obtained from transgenic rabbit milk (FVII HPTG, purity: 98%) was diluted in running buffer (50 mM Tris, 10 mM CaCl₂, 4 mM MgCl₂, pH 7.4) so as to obtain a sample with an FVII concentration of 200 mM.

The sample was injected onto the chip (solid substrate) containing the Mapt2 aptamer immobilized by means of a biotin-streptavidin interaction. Next, a buffer containing 50% propylene glycol was injected onto the solid substrate. All the injections were carried out with a flow rate of 30 μl/min for 60 sec after the injection. After the injection with the buffer containing 50% propylene glycol, elution buffer (10 mM EDTA) was then injected for 75 sec with a flow rate of 30 μl/min in order to detach the FVII HPTG from the aptamer.

These analyses are carried out with the RPS Biacore T100 apparatus (GE). The modeling of the interactions recorded is carried out by means of the Biaevaluation software (GE).

The curves of binding of the Mapt2 immobilized aptamer to the transgenic human FVII were calculated with the dedicated module of the Biacore® control software, version 1.2.

The results of binding of the Mapt2 aptamer to human FVII made it possible to determine that the binding of the Mapt2 aptamer to human FVII is not detrimentally modified by the injection of the buffer containing propylene glycol.

### B. Results

The Results are Illustrated in FIG. 9.

1-16. (canceled)

17. An affinity substrate for selectively binding a coagulation protein, comprising a solid substrate material on which nucleic aptamers which bind specifically to said coagulation protein are immobilized.

18. The affinity substrate as claimed in claim 17, wherein said nucleic aptamers consist of deoxyribonucleic aptamers.

19. The affinity substrate as claimed in claim 17, wherein said nucleic aptamers are included in the structure of a compound of formula (I) below:
[FIX]ₓ-[SPAC]_{y}-[APT] (I),
in which:
[FIX] signifies a compound for immobilization on a substrate,
[SPAC] signifies a spacer chain,
[APT] signifies a nucleic acid which binds specifically to a coagulation protein,
x is an integer equal to 0 or 1, and
y is an integer equal to 0 or 1.

20. The affinity substrate as claimed in claim 17, wherein the coagulation protein is chosen from factor I (fibrinogen), factor II (prothrombin), factor V (proaccelerin), factor VII (proconvertin), factor VIII (anti-hemophilic factor A), factor IX (anti-hemophilic factor B), factor X (Stuart factor), factor XI (Rosenthal factor or PTA), factor XII (Hageman factor), factor XIII (fibrin-stabilizing factor or FSF), PK (Prekallikrein), HMWK (high-molecular-weight kininogen), tissue thromboplastin, heparin cofactor II (HCII), protein C (PC), thrombomodulin (TM), protein S (PS), von Willebrand factor (vWF) and tissue factor pathway inhibitor (TFPI), or else tissue factors.

21. The affinity substrate as claimed in claim 17, wherein the solid substrate material is chosen from resins, polymer beads, magnetic beads, paramagnetic beads, substrate materials of filter membranes, and polymer materials.

22. A method for immobilizing a coagulation protein on a substrate, comprising a step during which a sample containing said coagulation protein is brought into contact with an affinity substrate as claimed in claim 17.

23. A method for purifying a coagulation protein, comprising the following steps:
a) bringing a sample containing a coagulation protein into contact with an affinity substrate as claimed in claim 17, in order to form complexes between (i) the nucleic aptamers immobilized on said affinity substrate and (ii) said coagulation protein, and
b) releasing the protein from the complexes formed in step a), and
c) recovering said coagulation protein in a purified form.

24. The method as claimed in claim 23, wherein said sample is chosen from human blood plasma, or a fraction thereof, or milk of a mammal that is transgenic for said coagulation protein, or a fraction thereof.

25. The method as claimed in claim 23, wherein the blood plasma protein is human.

26. The method as claimed in claim 25, wherein the sample comprises at least one nonhuman blood plasma protein.

27. The method as claimed in claim 26, wherein said human blood plasma protein is homologous to said nonhuman plasma protein.

28. The method as claimed in claim 27, wherein said human blood plasma protein is the homolog of said nonhuman plasma protein.

29. The method as claimed in claim 23, wherein step b) is carried out by bringing the affinity substrate into contact with an elution buffer containing a divalent-ion-chelating agent, preferably EDTA.

30. A purified composition of a recombinant human coagulation protein comprising at least 99.9% by weight of said recombinant human protein and which is substantially free of nonhuman proteins.

31. A pharmaceutical composition comprising a purified composition of a recombinant human coagulation protein as claimed in claim 30.

32. The affinity substrate as claimed in claim 18, wherein said nucleic aptamers are included in the structure of a compound of formula (I) below:
[FIX]ₓ-[SPAC]_{y}-[APT] (I),
in which:
[FIX] signifies a compound for immobilization on a substrate,
[SPAC] signifies a spacer chain,
[APT] signifies a nucleic acid which binds specifically to a coagulation protein,
x is an integer equal to 0 or 1, and
y is an integer equal to 0 or 1.

33. The method as claimed in claim 24, wherein the blood plasma protein is human.


SEQUENCE LISTING (101 sequences)

SEQ ID NO: 1 - DNA, 18 bp, artificial sequence
  aptamer
gggagatagccacgacct

SEQ ID NO: 2 - DNA, 18 bp, artificial sequence
  aptamer
tccaggctgtgcgaaagc

SEQ ID NO: 3 - DNA, 44 bp, artificial sequence
  aptamer
ccgcacaccacgcgcattagcccgcgcacacgacttgaagtagc

SEQ ID NO: 4 - DNA, 44 bp, artificial sequence; identical to SEQ ID NO: 3
  aptamer

SEQ ID NO: 5 - DNA, 43 bp, artificial sequence
  aptamer
cgcacaccacgcgcattagcccgcgcacacgacttgaagtagc

SEQ ID NO: 6 - DNA, 44 bp, artificial sequence
  aptamer
ccgcacaccacgcgcatgaacccgcgcacacgacttgaagtagc

SEQ ID NO: 7 - DNA, 44 bp, artificial sequence; identical to SEQ ID NO: 6
  aptamer

SEQ ID NO: 8 - DNA, 44 bp, artificial sequence; identical to SEQ ID NO: 6
  aptamer

SEQ ID NO: 9 - DNA, 44 bp, artificial sequence; identical to SEQ ID NO: 6
  aptamer

SEQ ID NO: 10 - DNA, 44 bp, artificial sequence
  aptamer
acgcacaccacgcgcatgaacccgcgcacacgacttgaagtagc

SEQ ID NO: 11 - DNA, 44 bp, artificial sequence
  aptamer
ccgcacaccacgcgcatgagcccgcgcacacgacttgaagtagc

SEQ ID NO: 12 - DNA, 44 bp, artificial sequence; identical to SEQ ID NO: 11
  aptamer

SEQ ID NO: 13 - DNA, 44 bp, artificial sequence; identical to SEQ ID NO: 11
  aptamer

SEQ ID NO: 14 - DNA, 45 bp, artificial sequence
  aptamer
gccgcacaccacgcgcatgagcccgcgcacacgacttgaagtagc

SEQ ID NO: 15 - DNA, 43 bp, artificial sequence
  aptamer
cgcacaccacgcgcatgagcccgcgcacacgacttgaagtagc

SEQ ID NO: 16 - DNA, 44 bp, artificial sequence
  aptamer
tcgcacaccacgcgcatgagcccgcgcacacgacttgaagtagc

SEQ ID NO: 17 - DNA, 44 bp, artificial sequence
  aptamer
acgcacaccacgcgcatgagcccgcgcacacgacttgaagtagc

SEQ ID NO: 18 - DNA, 44 bp, artificial sequence; identical to SEQ ID NO: 17
  aptamer

SEQ ID NO: 19 - DNA, 43 bp, artificial sequence
  aptamer
cgcacaccacgcgcatgancccgcgcacacgacttgaagtagc

SEQ ID NO: 20 - DNA, 44 bp, artificial sequence
  aptamer
ccgcacaccacgcgcataatcccgcgcacacgacttgaagtagc

SEQ ID NO: 21 - DNA, 44 bp, artificial sequence
  aptamer
ccgcacaccacgcgcatgaccccgcgcacacgacttgaagtagc

SEQ ID NO: 22 - DNA, 44 bp, artificial sequence; identical to SEQ ID NO: 21
  aptamer

SEQ ID NO: 23 - DNA, 43 bp, artificial sequence
  aptamer
cgcacaccacgcgcgtgaccccgcgcacacgacttgaagtagc

SEQ ID NO: 24 - DNA, 44 bp, artificial sequence
  aptamer
ccgcacaccacgcgcattaacccgcgcacacggcttggagtagc

SEQ ID NO: 25 - DNA, 44 bp, artificial sequence
  aptamer
tcgcacactacgcgcatgaacccgcgcacacgacttgaagtagc

SEQ ID NO: 26 - DNA, 44 bp, artificial sequence; identical to SEQ ID NO: 25
  aptamer

SEQ ID NO: 27 - DNA, 43 bp, artificial sequence
  aptamer
cgcacactacgcgcatgaacccgcgcacacgacttgaagtagc

SEQ ID NO: 28 - DNA, 45 bp, artificial sequence
  aptamer
gccgcacactacgcgcatgagcccgcgcacacgacttgaagtagc

SEQ ID NO: 29 - DNA, 44 bp, artificial sequence
  aptamer
tcgcacactacgcgcatgagcccgcgcacacgacttgaagtagc

SEQ ID NO: 30 - DNA, 44 bp, artificial sequence
  aptamer
ccgcacactacgcgcatgatcccgcgcacacgacttgaagtagc

SEQ ID NO: 31 - DNA, 44 bp, artificial sequence; identical to SEQ ID NO: 30
  aptamer

SEQ ID NO: 32 - DNA, 44 bp, artificial sequence
  aptamer
ctgcacactacgcgcatgatcccgcgcacacgacttgaagtagc

SEQ ID NO: 33 - DNA, 44 bp, artificial sequence
  aptamer
ccgcacgctacgcgcatgaacccgcgcacacgacttgaagtagc

SEQ ID NO: 34 - DNA, 44 bp, artificial sequence
  aptamer
ccgcacactacgcgcacgaacccgcgcacacgacttgaagtagc

SEQ ID NO: 35 - DNA, 44 bp, artificial sequence
  aptamer
tcgcacactacgcgcatgaccccgcgcacacgacttgaagtagc

SEQ ID NO: 36 - DNA, 43 bp, artificial sequence
  aptamer
agcacaccacgcgcataaacccgcgcacacgacttgaagtagc

SEQ ID NO: 37 - DNA, 44 bp, artificial sequence
  aptamer
gagcacaccacgcgcatgaacccgcgcacacgacttgaagtagc

SEQ ID NO: 38 - DNA, 46 bp, artificial sequence
  aptamer
gctagcacaccacgcgcatgaacccgcgcacacgacttgaagtacc

SEQ ID NO: 39 - DNA, 44 bp, artificial sequence
  aptamer
acgcacaccacgcgcatgaacccgcgcacatgacttgaagtagc

SEQ ID NO: 40 - DNA, 44 bp, artificial sequence
  aptamer
ccgcacaccacgcgcattagcccgcgcacacgacttgaagttaa

SEQ ID NO: 41 - DNA, 44 bp, artificial sequence
  aptamer
ccggagacgcgcagctcctatacatgcgcacatgacttgaagtc

SEQ ID NO: 42 - DNA, 44 bp, artificial sequence; identical to SEQ ID NO: 41
  aptamer

SEQ ID NO: 43 - DNA, 44 bp, artificial sequence
  aptamer
cttgagacgcgcatttgctatacatgcgcacgtgacttgaagtc

SEQ ID NO: 44 - DNA, 44 bp, artificial sequence; identical to SEQ ID NO: 43
  aptamer

SEQ ID NO: 45 - DNA, 43 bp, artificial sequence
  aptamer
ctgagacgcgcanttgctatacatgcgcacatgacttgaagtc

SEQ ID NO: 46 - DNA, 46 bp, artificial sequence
  aptamer
ctggagacgcgcagttgctgtacatgcgcacatgacttgaagtagc

SEQ ID NO: 47 - DNA, 44 bp, artificial sequence
  aptamer
gtggagacgcgcatttgctgtacatgcgcacatgactagaagtc

SEQ ID NO: 48 - DNA, 44 bp, artificial sequence
  aptamer
caggagacgcgcatttgttgtacatgcgcacatgactagaagtc

SEQ ID NO: 49 - DNA, 44 bp, artificial sequence
  aptamer
ctggagacgcgcagttgttgtacacgcgcacatgactagaagtc

SEQ ID NO: 50 - DNA, 44 bp, artificial sequence
  aptamer
ctggagacgcgcagttgctacacacgcgcacatgactagaagtc

SEQ ID NO: 51 - DNA, 44 bp, artificial sequence
  aptamer
ctggagacgcgcacttcctttacatgcgcacatgactagaagtc

SEQ ID NO: 52 - DNA, 44 bp, artificial sequence
  aptamer
ctggagacgcgcagttgctttacatgcgcacatgactagaagtc

SEQ ID NO: 53 - DNA, 44 bp, artificial sequence
  aptamer
ctggagacgcgcagttgctgtacatgcgcacatgactagaagtc

SEQ ID NO: 54 - DNA, 44 bp, artificial sequence; identical to SEQ ID NO: 53
  aptamer

SEQ ID NO: 55 - DNA, 44 bp, artificial sequence
  aptamer
ctggggacgcgcagttgctgtacatgcgcacatgactagaagtc

SEQ ID NO: 56 - DNA, 44 bp, artificial sequence
  aptamer
ctggagacgcgcagttaccatacgtgcgcacatgactagaagtc

SEQ ID NO: 57 - DNA, 44 bp, artificial sequence
  aptamer
ctggagacgcgcagttgctgtacgtgcgcacatgactagaagtc

SEQ ID NO: 58 - DNA, 44 bp, artificial sequence
  aptamer
agtctccatgttgcgaaccgaatggtaaggggatcgtacactac

SEQ ID NO: 59 - DNA, 44 bp, artificial sequence; identical to SEQ ID NO: 58
  aptamer

SEQ ID NO: 60 - DNA, 44 bp, artificial sequence
  aptamer
agactccatgatgcgaaccgaatggtaagggaatcgtacaatac

SEQ ID NO: 61 - DNA, 44 bp, artificial sequence; identical to SEQ ID NO: 60
  aptamer

SEQ ID NO: 62 - DNA, 44 bp, artificial sequence
  aptamer
agactccattatgcgaaccgaatggtaagggaatcgtacaatac

SEQ ID NO: 63 - DNA, 44 bp, artificial sequence
  aptamer
agactccatgatgcgaaccgaatggtaagggaatcgtacagtac

SEQ ID NO: 64 - DNA, 44 bp, artificial sequence; identical to SEQ ID NO: 63
  aptamer

SEQ ID NO: 65 - DNA, 44 bp, artificial sequence
  aptamer
agactccatgatgcgaaccgaatggtaagggaatcgtacaatgc

SEQ ID NO: 66 - DNA, 44 bp, artificial sequence; identical to SEQ ID NO: 65
  aptamer

SEQ ID NO: 67 - DNA, 44 bp, artificial sequence; identical to SEQ ID NO: 65
  aptamer

SEQ ID NO: 68 - DNA, 44 bp, artificial sequence
  aptamer
agactccatgatgcgaaccgaatggtaagggaatcgtacgacac

SEQ ID NO: 69 - DNA, 44 bp, artificial sequence
  aptamer
agactccatggtgcgaaccgaatggtaagggaatcgtacaacac

SEQ ID NO: 70 - DNA, 44 bp, artificial sequence
  aptamer
tgactccatgatgcgaaccgaatggtaagggaatcgtacaacac

SEQ ID NO: 71 - DNA, 44 bp, artificial sequence
  aptamer
agactccatgatgcgaaccgaatggttagggaatcgtacaatac

SEQ ID NO: 72 - DNA, 44 bp, artificial sequence; identical to SEQ ID NO: 71
  aptamer

SEQ ID NO: 73 - DNA, 44 bp, artificial sequence
  aptamer
agactccatgatgcgaaccgaatggttagggaatcgtacaacac

SEQ ID NO: 74 - DNA, 44 bp, artificial sequence
  aptamer
agaccccatgatgcgaaccgaatggttagggaatcgtacagtac

SEQ ID NO: 75 - DNA, 44 bp, artificial sequence
  aptamer
agaccccctgatgcgaaccgaatggttagggaatcgtacaatac

SEQ ID NO: 76 - DNA, 44 bp, artificial sequence
  aptamer
agactccatgatgcgaaccgaatggtaagggaatcgcactgtac

SEQ ID NO: 77 - DNA, 44 bp, artificial sequence
  aptamer
agactccaagatgcgaaccgaatggtaagggaatcgtacagtac

SEQ ID NO: 78 - DNA, 44 bp, artificial sequence
  aptamer
agaccccgtgatgcgaaccgaatggtaagggaatcgcacaatac

SEQ ID NO: 79 - DNA, 44 bp, artificial sequence
  aptamer
agactccccgatgcgaaccgaatggtaagggaatcgtacgatgc

SEQ ID NO: 80 - DNA, 44 bp, artificial sequence
  aptamer
agaccccatgacgcgaaccgaatggtaagggaatcgtacaatgc

SEQ ID NO: 81 - DNA, 44 bp, artificial sequence
  aptamer
agaccccatgatgcgaaccgaatggtaagggaatcgtactacac

SEQ ID NO: 82 - DNA, 44 bp, artificial sequence
  aptamer
agaccccatgatgcgaaccgaatggtaaggggatcgtacaaccc

SEQ ID NO: 83 - DNA, 43 bp, artificial sequence
  aptamer
agactccatgatgcgaaccgaatggtaagggaatcatacaccc

SEQ ID NO: 84 - DNA, 44 bp, artificial sequence
  aptamer
agactccatgatgctaaccgaatggttagggaatcgtacgacgc

SEQ ID NO: 85 - DNA, 45 bp, artificial sequence
  aptamer
atgcagccagccgcagtgtaagtgaatgcagacatggtctaagtg

SEQ ID NO: 86 - DNA, 81 bp, artificial sequence
  aptamer
gggagatagccacgacctatgcagccagccgcagtgtaagtgaatgcagacatggtctaagtgtccaggctgtgcgaaagc

SEQ ID NO: 87 - DNA, 43 bp, artificial sequence
  aptamer
taagccgctgcgtattatagctgaatgcccctaatgggaagtg

SEQ ID NO: 88 - DNA, 44 bp, artificial sequence
  aptamer
catgccgctgcgtattatagctgaatgccccacaatgggaagtg

SEQ ID NO: 89 - DNA, 44 bp, artificial sequence
  aptamer
caagccgctgcgtattatagctgaatgccccataatgggaagtg

SEQ ID NO: 90 - DNA, 43 bp, artificial sequence
  aptamer
caagccgctgcgtatttagctgaatgccccctaatgggaagtg

SEQ ID NO: 91 - DNA, 44 bp, artificial sequence
  aptamer
ccagccgctgcgtatcatagctgaatgccccataatgggaagtg

SEQ ID NO: 92 - DNA, 43 bp, artificial sequence
  aptamer
caagccgctgcgtaatatagctgaatgccccataatggaagtg

SEQ ID NO: 93 - DNA, 44 bp, artificial sequence
  aptamer
caagcctctgcgtattatagctgaatgctccttaatggtaagtg

SEQ ID NO: 94 - DNA, 44 bp, artificial sequence
  aptamer
caagccgctgcgtattatagctgaatgctccttaatggtaagtg

SEQ ID NO: 95 - DNA, 44 bp, artificial sequence
  aptamer
ccaggcgctgcgtatcatagctgaatgctccttaacggtaagtg

SEQ ID NO: 96 - DNA, 44 bp, artificial sequence
  aptamer
caagccgctgcgttttatagctgaatgctcctcaatggtaagtg

SEQ ID NO: 97 - DNA, 44 bp, artificial sequence
  aptamer
caagccgctgcgttttatagctgaatgctccatcttggtaagtg

SEQ ID NO: 98 - DNA, 44 bp, artificial sequence
  aptamer
gtgcagccagccgcagtntaagtgagctgattgaagcatgaggg

SEQ ID NO: 99 - DNA, 45 bp, artificial sequence; identical to SEQ ID NO: 85
  aptamer

SEQ ID NO: 100 - DNA, 45 bp, artificial sequence
  aptamer
ccagcagagccgcagtttcagtgaatgcagatcatggtctaagtg

SEQ ID NO: 101 - DNA, 80 bp, artificial sequence
  aptamer
gggagatagccacgacctcgcacatgacttgaagttaaacgcgaattacaaacccagccccctccaggctgtgcgaaagc